(19) United States
(12) Patent Application Publication
(10) Pub. No.: US 2025/0312158 A1
(43) Pub. Date: Oct. 9, 2025

(54) MEDICAL DEVICE HOLDING STRUCTURE AND MOUNT

(71) Applicant: Terumo Kabushiki Kaisha, Tokyo (JP)

(72) Inventors: Koichi HAYAKAWA, Ashigarakami-gun (JP); Yoshihiko HAMAGUCHI, Nagoya-shi (JP)

(73) Assignee: Terumo Kabushiki Kaisha, Tokyo (JP)

(21) Appl. No.: 19/244,855

(22) Filed: Jun. 20, 2025

Related U.S. Application Data
(63) Continuation of application No. PCT/JP2023/046056, filed on Dec. 22, 2023.

(30) Foreign Application Priority Data
Dec. 23, 2022 (JP) 2022-206596

(51) Int. Cl. A61F 2/24

(52) U.S. Cl. CPC A61F 2/2481; A61F 2002/2484

(57) ABSTRACT

A medical device holding structure includes a medical device and a mount having a holding surface for holding the medical device, in which the mount includes a cover portion that is continuous with a mount body and that covers a first expansion body of the medical device in a state where a first inner member moves in a distal direction with respect to an outer cylinder of the medical device and the first expansion body is unfolded. The cover portion has a bent shape folded back in such a way as to cover the first and second expansion bodies of the medical device.

### CROSS-REFERENCES TO RELATED APPLICATIONS

This application is a continuation of International Application No. PCT/JP2023/046056 filed on Dec. 22, 2023, which claims priority to Japanese Patent Application No. 2022-206596 filed on Dec. 23, 2022, the entire content of both of which is incorporated herein by reference.

### TECHNOLOGICAL FIELD

The present invention generally relates to a medical device holding structure capable of holding or configured to hold a medical device and a mount.

### BACKGROUND DISCUSSION

JP 2009-000511 A discloses a medical device for transferring a medical sheet (cell sheet) for use in, for example, organ transplantation to a treatment site of a living body. The medical device includes an outer cylinder, a shaft, and a support portion provided at a distal portion of the shaft. The support portion includes a sheet support on which the medical sheet is placed. The medical sheet is transferred from the support portion to the treatment site by sliding the transfer instrument while pressing an upper surface of the medical sheet placed on a support surface of the sheet support with forceps or the like.

### SUMMARY

In the above-described conventional technique, when the sheet support is formed of a flexible resin material and kept deformed for an extended period of time to be housed and stored in the outer cylinder, the deformation of the sheet support might become permanent. When the sheet support is extended out of the outer cylinder and unfolded to use the medical device, there is a concern that the sheet support does not unfold in a predetermined shape. In addition, in a medical device including another expansion body, there is a concern that the expansion body is deformed by an external force.

(1) An aspect of the disclosure here is a medical device holding structure including a medical device and a mount that holds the medical device, in which the medical device includes a shaft and an expandable and contractible expansion body disposed at a distal portion of the shaft, the mount includes a mount body having a holding surface for holding the medical device, and a cover portion that is continuous with the mount body and that covers the expansion body of the medical device, the cover portion has a bent shape folded back in such a way as to cover the expansion body, and a housing portion in which the expansion body is housed is formed inside the cover portion.

With this medical device holding structure, since the expansion body can be covered and protected by the cover portion, deformation of the expansion body due to application of an external force is prevented. As a result, it is possible to store the expansion body of the medical device while protecting the expansion body in the medical device holding structure.

(2) In the medical device holding structure according to (1), the mount may include a support piece raised on the holding surface of the mount body to hold the medical device, and when the cover portion is unfolded in a direction away from the expansion body, the support piece may tilt toward the holding surface.

With this configuration, when the cover portion is unfolded and the medical device is taken out, the support piece tilts toward the holding surface, so that the medical device is easily taken out.

(3) In the medical device holding structure according to (2), the mount may include a plurality of device holding portions capable of holding a plurality of the medical devices, and the plurality of device holding portions may include a plurality of the support pieces.

With this configuration, a plurality of medical devices can be held on a single mount, which is efficient.

(4) The medical device holding structure according to (3) may further include a separation portion that can be used to separate one device holding portion and another device holding portion between two adjacent device holding portions among the plurality of device holding portions.

With this configuration, in a state where the medical devices are held by the plurality of device holding portions, each medical device can be separated using the separation portion at a time of use.

(5) In the medical device holding structure according to any one of (1) to (4), the mount may include a cover separation portion that can be used to separate the cover portion from the mount body, and the cover separation portion may have a break line for breaking the cover portion from the mount body.

With this configuration, when the medical device is taken out from the mount, it is possible to prevent contact between the expansion body of the medical device and the cover portion by separating and removing the unfolded cover portion from the mount body via the break line of the cover separation portion.

(6) In the medical device holding structure according to any one of (1) to (5), the cover portion may have an opening penetrating the cover portion, and the expansion body may be visually recognizable from an outside of the cover portion through the opening.

With this configuration, the user can visually recognize the expansion body from the outside of the cover portion in a state where the expansion body is housed inside the cover portion.

(7) The medical device holding structure according to any one of (1) to (6), in which the medical device may include an outer cylinder and an inner member disposed inside the outer cylinder in such a way as to be movable along an axial direction of the outer cylinder, the inner member may include the shaft inserted into the outer cylinder and the expansion body disposed at a distal portion of the shaft, the expansion body may be housed in the outer cylinder in a contracted state at a first position where the inner member is moved in a proximal direction with respect to the outer cylinder such that the expansion body is housed in the outer cylinder, the expansion body may be unfolded by being exposed from the outer cylinder in a distal direction at a second position where the inner member is moved in the distal direction with respect to the outer cylinder such that the expansion body protrudes from a distal-end opening of the outer cylinder, and the expansion body of the medical device may be covered with the cover portion in a state where the inner member is located at the second position with respect to the outer cylinder and the expansion body is unfolded.

With this configuration, since the expansion body can be covered and protected by the cover portion in a state where the expansion body is unfolded outside the outer cylinder, it is possible to prevent deformation due to housing in the outer cylinder from remaining in the expansion body. As a result, it is possible to protect and store the expansion body of the medical device in the medical device holding structure, and it is possible to reliably and stably unfold the expansion body when the expansion body protrudes from the outer cylinder at the time of using the medical device.

(8) The medical device holding structure according to (7), in which the expansion body may be formed of a flexible sheet having a front surface including a support surface capable of holding a medical sheet and a back surface opposite to the front surface, the expansion body may include a pair of protrusions protruding upward from both sides, in a width direction, of the support surface orthogonal to a moving direction of the shaft, and at the first position, the expansion body may be housed in the outer cylinder in a state of being curved and deformed such that both sides, in the width direction, of the expansion body on the back surface come into contact with each other.

(9) Another aspect of the disclosure is a mount for holding a medical device including a shaft and an expansion body disposed at a distal portion of the shaft, the mount including a mount body having a holding surface for holding the medical device, and a cover portion that is continuous with the mount body and that covers the expansion body of the medical device, in which the cover portion is folded back in a state where the medical device is held by the mount body, so that the cover portion can cover the expansion body of the medical device.

With this mount, since the cover portion can cover and protect the expansion body, deformation of the expansion body due to application of an external force is prevented.

(10) The mount according to (9) may further include a plurality of device holding portions capable of holding a plurality of the medical devices, in which the plurality of device holding portions may include a plurality of support pieces.

With this configuration, a plurality of medical devices can be held on a single mount, which is efficient.

(11) The mount according to (10) may further include a separation portion that can be used to separate one device holding portion and another device holding portion from each other between two adjacent device holding portions among the plurality of device holding portions.

With this configuration, in a state where the medical devices are held in the device holding portions, each medical device to be used can be separated via the separation portion.

(12) In the medical device holding structure according to any one of (9) to (11), the mount may include a cover separation portion that can be used to separate the cover portion from the mount body, and the cover separation portion may have a break line for breaking the cover portion from the mount body.

With this configuration, when the medical device is taken out from the mount, contact between the expansion body of the medical device and the cover portion is prevented by separating and removing the unfolded cover portion from the mount body via the cover separation portion.

Because the expansion body can be covered and protected by the cover portion, deformation of the expansion body due to application of an external force can be prevented. This makes it possible to protect the expansion body in the medical device holding structure.

According to another aspect a medical device holding structure comprises a medical device and a mount. The medical device includes an elongated shaft having a distal portion, with the medical device also including an expandable and contractible expansion body positioned at the distal portion of the shaft and extending away from the distal portion of the elongated shaft. The mount has oppositely disposed first and second surfaces, with one end portion of the mount being folded back upon itself so that the first surface of the mount faces a housing portion. The one end portion of the mount that is folded back upon itself defines a cover portion of the mount, with the mount including an elongated mount body that extends from the cover portion to an opposite end of the mount that is opposite the one end portion. The elongated shaft of the medical device overlies the first surface of the mount so that the first surface of the mount in the elongated mount body faces the elongated shaft while the second surface of the mount in the elongated mount body faces away from the elongated shaft. The expandable and contractible expansion body is positioned in the housing portion so that the expandable and contractible expansion body is covered by the cover portion.

### BRIEF DESCRIPTION OF THE DRAWINGS

FIG. 1 is an overall plan view of a medical device package according to an embodiment representing an example of the medical device package.

FIG. 2 is a perspective view of a medical device (transfer instrument) housed in the medical device package illustrated in FIG. 1.

FIG. 3 is an exploded perspective view of the transfer instrument illustrated in FIG. 1.

FIG. 4 is a plan view of a distal portion of the transfer instrument illustrated in FIG. 1.

FIG. 5 is a vertical cross-sectional view taken along the section line V-V in FIG. 4.

FIG. 6 is a transverse cross-sectional view taken along the section line VI-VI in FIG. 4.

FIG. 7 is a developed view of a mount constituting a medical device holding structure.

FIG. 8 is an overall side view of the medical device holding structure illustrated in FIG. 1.

FIG. 9A is an enlarged plan view of the vicinity of first support pieces of the medical device holding structure, and FIG. 9B is an enlarged plan view of the vicinity of second support pieces of the medical device holding structure.

FIG. 10A is a cross-sectional view taken along the section line XA-XA in FIG. 8, and FIG. 10B is a cross-sectional view taken along the section line XB-XB in FIG. 8.

FIG. 11 is an enlarged plan view illustrating the vicinity of a cover portion in FIG. 1.

FIG. 12 is an enlarged side view of a medical device holding structure in which a medical device according to a modification is housed.

FIG. 13 is an overall plan view of the medical device holding structure in a state in which the cover portion is unfolded.

FIG. 14 is an overall side view of the medical device holding structure illustrated in FIG. 13.

FIG. 15 is a flowchart illustrating a procedure of a method for transferring a medical sheet using the medical device in FIG. 2.

FIG. 16 is a first explanatory diagram of a sheet placing step.

FIG. 17 is a second explanatory diagram of the sheet placing step.

FIG. 18 is an explanatory diagram of a housing step.

FIG. 19 is a transverse cross-sectional view taken along the section line XIX-XIX in FIG. 18.

FIG. 20 is a transverse cross-sectional view taken along the section line XX-XX in FIG. 18.

FIG. 21 is an explanatory diagram of a positioning step.

FIG. 22 is an explanatory diagram of an unfolding step.

FIG. 23 is an explanatory diagram of a moving step.

FIG. 24 is an explanatory diagram of a withdrawing step.

FIG. 25 is a cross-sectional view of a transfer instrument including a movement restriction portion according to a first modification.

FIG. 26A is a plan view of a distal portion of a transfer instrument that is a movement restriction portion according to a second modification, and FIG. 26B is a plan view illustrating a state in which a first expansion body of the transfer instrument in FIG. 26A is housed in an outer cylinder.

FIG. 27 is a plan view of a distal portion of a transfer instrument including a first expansion body according to a third modification.

FIG. 28 is a cross-sectional view taken along the section line XXVIII-XXVIII in FIG. 27.

FIG. 29 is an enlarged perspective view of a distal portion of a transfer instrument including a pressing body according to a fourth modification.

FIG. 30 is an enlarged perspective view of a distal portion of a transfer instrument including a pressing body according to a fifth modification.

FIG. 31 is a cross-sectional view of a distal portion of the transfer instrument illustrated in FIG. 30.

### DETAILED DESCRIPTION

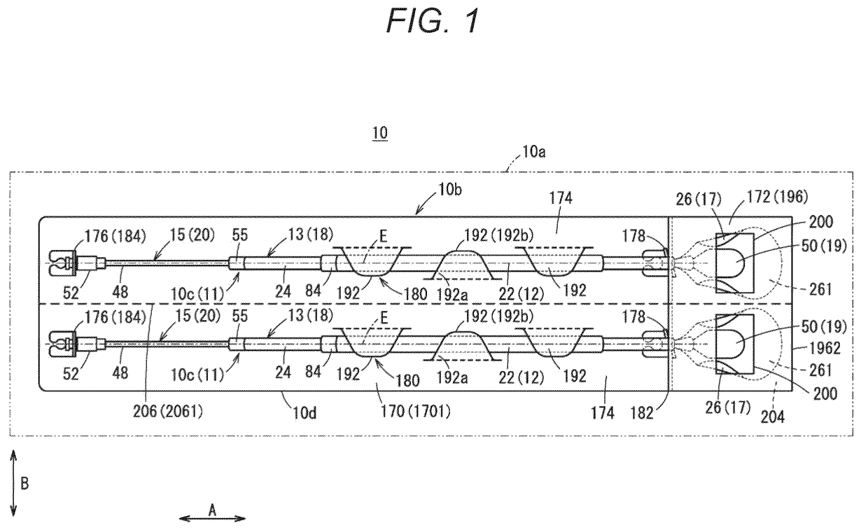

As illustrated in FIG. 1, a medical device package 10 according to the present embodiment includes a packaging container 10a and a medical device holding structure 10b housed inside the packaging container 10a. The packaging container 10a has, for example, a transparent and sealable bag shape. The medical device holding structure 10b is sealed inside the packaging container 10a.

The medical device holding structure 10b, representing an example of the new medical device holding structure disclosed here, includes medical devices 10c and a mount 10d that holds the medical devices 10c.

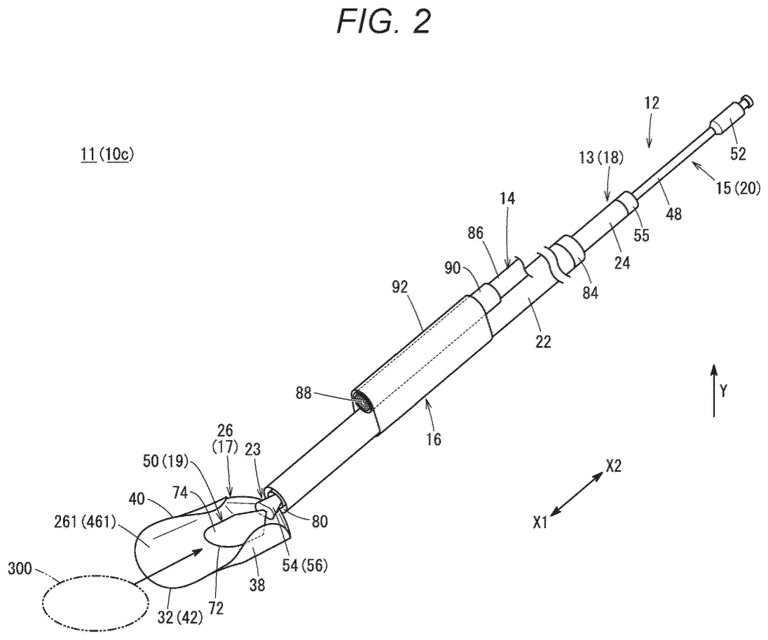

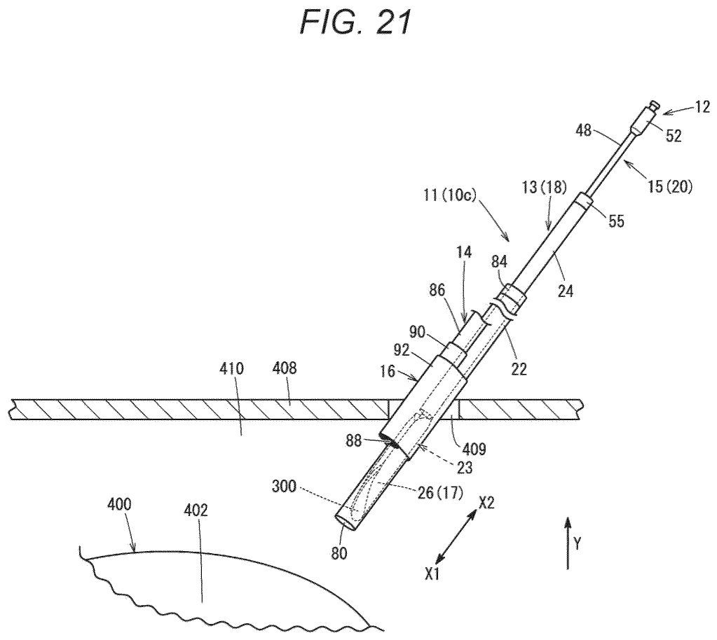

As illustrated in FIG. 2, each medical device 10c is a transfer instrument 11 for transferring a medical sheet 300 to a treatment site of a living body. The medical device 10c is not limited to the transfer instrument 11. For example, the medical device 10c may be a treatment instrument for treating a treatment site of a living body. The transfer instrument 11 is used for, for example, treatment of severe heart failure caused by ischemic heart disease. In this case, the medical sheet 300 is transplanted to a recipient site 402 of a heart 400 (the treatment site of the living body) (see FIGS. 21 to 24). The transfer instrument 11 is capable of attaching a plurality of the medical sheets 300 to the recipient site 402.

Examples of such a medical sheet 300 include pharmaceutical products and regenerative medicine products for medical use, the medical device 10c, and the like. The medical sheet 300 is formed in a sheet shape such as a film shape or a membrane shape (gel object). Fibrin or the like may be applied to the medical sheet 300 for reinforcement. Examples of the regenerative medicine products including cells include a cell sheet (sheet-shaped cell culture), a spheroid, and the like. It is possible to form the cell sheet by culturing autologous cells or allogenic cells. The cells constituting the cell sheet include, for example, somatic stem cells (adult stem cells), mesenchymal stem cells, or iPS cells (induced pluripotent stem cells)-derived cardiomyocytes. Examples of the somatic stem cells preferably include skeletal myoblast cells (myoblast cells).

The medical sheet 300 may contain a tissue adhesive, a local anesthetic, or the like. The medical sheet 300 has a thickness of, for example, about 100 μm, and has a diameter of, for example, about 60 mm. The thickness and the diameter (size) of the medical sheet 300, however, may be set as desired.

The medical sheet 300 may be a sheet to be transplanted to an organ (for example, lung, liver, pancreas, kidney, small intestine, esophagus, or the like) other than the heart 400, instead. Alternatively, the medical sheet 300 may be, for example, an anti-adhesion sheet as long as the sheet is for medical use.

As illustrated in FIG. 2, the transfer instrument 11 includes an instrument body 12, an endoscope 14, and a fixing member 16. The instrument body 12 includes a first inner member 13, a second inner member 15, an outer cylinder 22, and a movement restriction portion 23. Note that the transfer instrument 11 is not limited to a configuration including the endoscope 14.

The first inner member 13 is disposed inside the outer cylinder 22 in such a way as to be movable along an axial direction of the outer cylinder 22. The first inner member 13 includes a first shaft 24 and a first expansion body 26.

The first shaft 24 is a tubular body (in the present embodiment, a circular tube member) having a first lumen 28. As illustrated, the first shaft 24 may be an elongated shaft. The first lumen 28 opens at a distal portion (an end in a direction of an arrow X1) of the first shaft 24 and opens at a proximal end (an end in a direction of an arrow X2) of the first shaft 24. The first shaft 24 is provided with, at the proximal end thereof, an airtight valve 55 that is in close contact with an outer peripheral surface of a second shaft 48. The first shaft 24 is not limited to a tubular body, and may be a body other than a tubular body, instead.

The first shaft 24 extends in the axial direction of the outer cylinder 22 and is disposed inside the outer cylinder 22 in such a way as to be movable along the axial direction. The first shaft 24 is composed of, for example, a resin material. Examples of a constituent material from which the first shaft 24 may be fabricated include, but not particularly limited to, polyethylene, polypropylene, fluororesin, polyethylene terephthalate, polymethyl methacrylate, a polyamide resin, polystyrene, polycarbonate, polyimide, polyetherimide, polyetheretherketone, polyvinyl chloride, an ABS resin, a polyamide elastomer, and a polyester elastomer. The first shaft 24 may be composed of a metal material.

The first shaft 24 may be flexible. The first shaft 24 may have a flexible tube portion capable of maintaining a bent shape. In this case, the first shaft 24 can be bent into any desired shape in a body cavity and is capable of maintaining the bent shape.

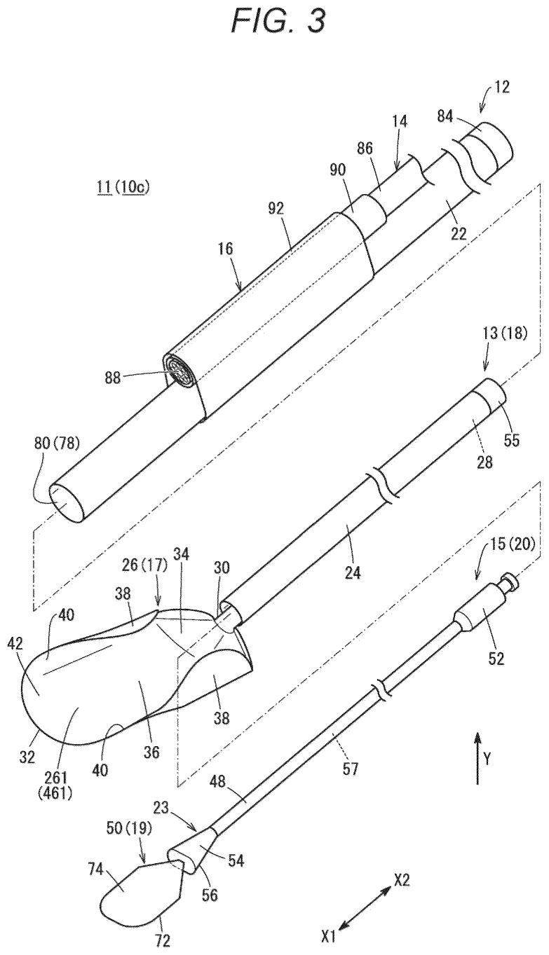

As illustrated in FIGS. 3 to 5, the first expansion body 26 is attached to the distal portion of the first shaft 24. The first expansion body 26 is composed of, for example, a resin material. In the present embodiment, the first expansion body 26 is a sheet-like first support portion 17 capable of holding the medical sheet 300. In the present embodiment, therefore, the first inner member 13 is a first carrier member 18 including the first shaft 24 and the first support portion 17. A flexible resin sheet member (film member) is bent into a predetermined shape to form the first expansion body 26. Alternatively, a sheet member is formed into a predetermined shape using a sheet forming die to form the first expansion body 26. It is preferable that the sheet member have, but not particularly limited to, a thickness of, for example, 100 μm or more and 200 μm or less. The first expansion body 26 includes a first joint 30 and a first support body 32.

A constituent material from which the first expansion body 26 may be made preferably has transparency, and examples of the constituent material include, but not particularly limited to, polyethylene, polycarbonate, polyamide, polystyrene, polypropylene, a polyacetal resin, polyimide, polyetherimide, polyetheretherketone, polyethylene terephthalate, and fluororesin. In addition, the first expansion body 26 may have a mesh shape.

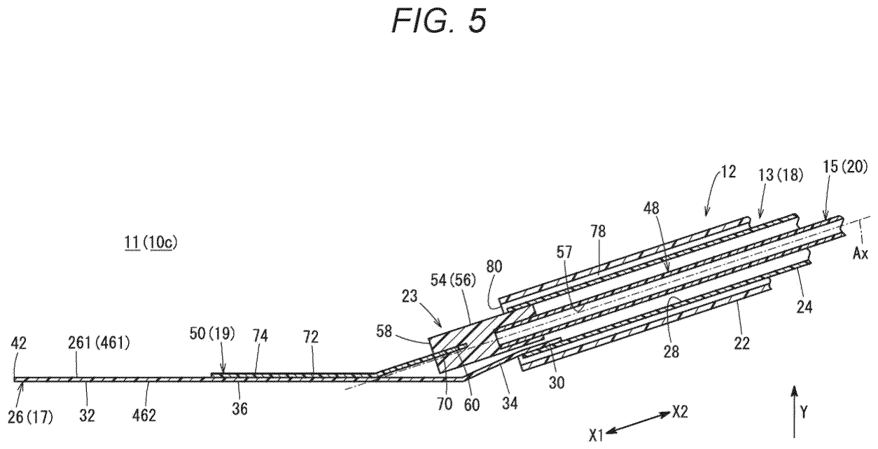

In FIG. 5, the first joint 30 is bonded to an inner peripheral surface of the distal portion of the first shaft 24 with an adhesive. Examples of the adhesive include, but are not particularly limited to, a UV adhesive, a hot-melt adhesive, and an instant adhesive (for example, a cyanoacrylate-based instant adhesive). The first joint 30 may be thermally fused to the inner peripheral surface of the first shaft 24.

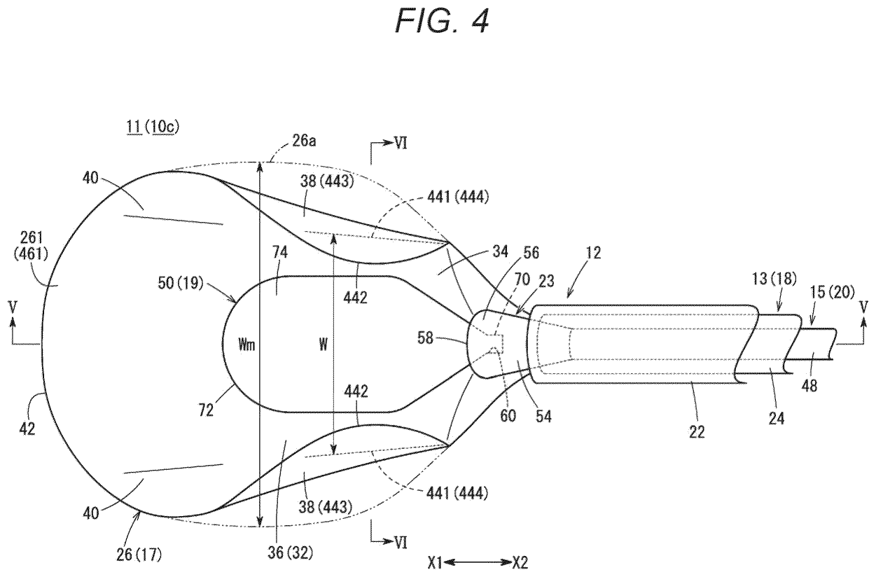

As illustrated in FIG. 4, the first support body 32 extends in a distal direction from the first joint 30 (see FIG. 5). The first support body 32 includes a proximal-end support portion 34, an intermediate support portion 36, a pair of first protrusions 38, a pair of second protrusions 40, and a distal-end support portion 42.

The proximal-end support portion 34 extends roughly along an axis Ax of the first shaft 24 from a distal end of the first joint 30 in the distal direction (the direction of the arrow X1) (see FIG. 5). The proximal-end support portion 34 is formed in such a way as to be wider in an extending direction thereof. In other words, both sides of the proximal-end support portion 34 in a width direction are tapered toward the first joint 30.

The intermediate support portion 36 intersects with the axis Ax of the first shaft 24 and extends from a distal end of the proximal-end support portion 34 toward a distal end of the first expansion body 26 (in the direction of arrow X1) (see FIG. 5). The intermediate support portion 36 is formed in a tapered shape in such a way as to become gradually narrower in width from the distal end toward the proximal end (the direction of the arrow X2).

In FIGS. 3 and 4, the pair of first protrusions 38 protrudes upward (in the direction of an arrow Y) from both sides of the intermediate support portion 36 in the width direction orthogonal to a moving direction of the first shaft 24 and inward in the width direction of the intermediate support portion 36. The pair of first protrusions 38 is connected to the proximal-end support portion 34.

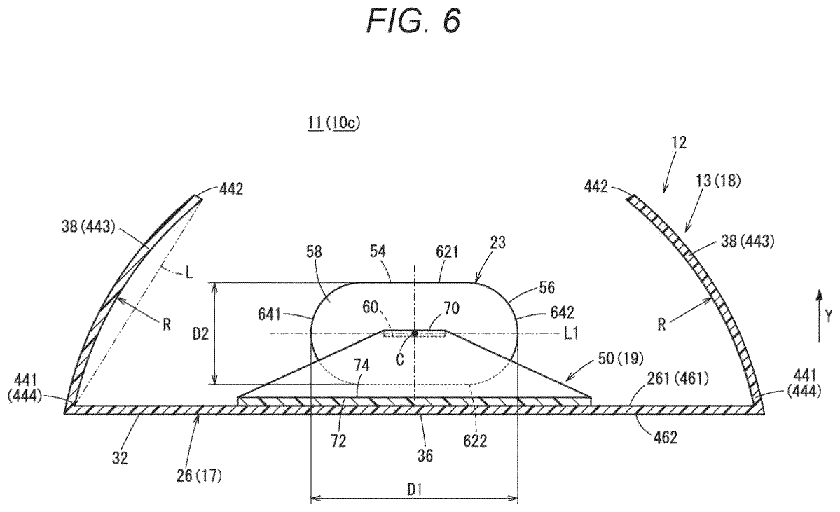

As illustrated in FIG. 6, each of the pair of first protrusions 38 includes a fixed end 441 connected to an upper surface (first support surface 261) of the intermediate support portion 36 and a free end 442 that is an end separated from the first support surface 261 in a protruding direction of the first protrusion 38.

In a cross section orthogonal to the protruding directions of the first protrusions 38 illustrated in FIG. 6 and orthogonal to the first support surface 261, each of cross sections of the first protrusions 38 has an intermediate portion 443 constituting a portion between the free end 442 and the fixed end 441. Each intermediate portion 443 has an arc shape bulging in a convex shape in a direction away from the first support surface 261. The cross section of the intermediate portion 443 is not limited to an arc shape having a single radius, and may have any arc shape. In other words, in each of the cross sections of the first protrusions 38 illustrated in FIG. 6, the intermediate portion 443 is disposed outside with respect to a line segment L connecting the fixed end 441 and the free end 442.

In the cross section orthogonal to the protruding directions of the first protrusions 38 illustrated in FIG. 6 and orthogonal to the first support surface 261, a curvature R of each intermediate portion 443 increases toward the proximal end (the direction of the arrow X2 in FIG. 4) of the first expansion body 26.

As illustrated in FIG. 4, the pair of second protrusions 40 is connected to distal ends of the pair of first protrusions 38. The pair of second protrusions 40 protrudes upward from both sides of the intermediate support portion 36 in the width direction and outward in the width direction of the intermediate support portion 36. The second protrusions 40 are formed with a smaller curvature than that of the first protrusions 38. The second protrusions 40 are lower in protrusion height from the first support surface 261 than the first protrusions 38.

The distal-end support portion 42 is connected to a distal end of the intermediate support portion 36 and distal ends of the pair of second protrusions 40. The distal-end support portion 42 protrudes in an arc shape in the distal direction (the direction of the arrow X1). That is, when viewed from a direction perpendicular to the first support surface 261 illustrated in FIG. 4, the distal-end support portion 42 of the first expansion body 26 has an arc shape connecting the pair of second protrusions 40 to each other.

Each of the pair of first protrusions 38 has a pair of bent portions 444 on a proximal side of the fixed end 441. Each of the pair of bent portions 444 causes a corresponding one of the pair of first protrusions 38 to bend from the first support surface 261 (intermediate support portion 36) of the first expansion body 26 (see FIG. 6). As illustrated in FIG. 4, when the bent portions 444 of the first expansion body 26 are bent to form the pair of first protrusions 38 bulging outward in a convex shape, it is preferable to bend, at the bent portions 444, both ends of a portion of a resin sheet material 26a (see a virtual line shape in FIG. 4) to be the first expansion body 26 having a maximum width Wm in the width direction.

When viewed from a direction orthogonal to the first support surface 261 illustrated in FIG. 4, an interval W in the width direction between the pair of bent portions 444 in the first expansion body 26 gradually decreases toward the proximal end (the direction of the arrow X2). That is, in the first support body 32, the pair of bent portions 444 is formed in a tapered shape in the proximal direction.

The first support body 32 has a front surface 461 that faces upward (the direction of the arrow Y) and that includes the first support surface 261, and a back surface 462 that is a surface opposite the front surface 461. The first support surface 261 is a flat surface continuous over an upper surface of the proximal-end support portion 34 and upper surfaces of the intermediate support portion 36 and the distal-end support portion 42. A lubricant may be applied to the first support surface 261 in order to allow a second expansion body 50, which will be described later, of a second carrier member 20 to smoothly slide on the first support surface 261.

As illustrated in FIG. 3, the second inner member 15 is disposed inside the first shaft 24 in such a way as to be movable along the axial direction of the first shaft 24. The second inner member 15 includes the second shaft 48 and the second expansion body 50. The second expansion body 50 is a sheet-like second support portion 19 capable of holding the medical sheet 300. The second inner member 15 includes a hub 52.

The second shaft 48 is a tubular body (in the present embodiment, a circular tube member) having a second lumen 57. As illustrated, the second 48 may be an elongated shaft. The second shaft 48 is longer than the first shaft 24 in the axial direction. The second shaft 48 is inserted into the first lumen 28 of the first shaft 24 (see FIGS. 2 and 5). In other words, a distal portion of the second shaft 48 protrudes in the distal direction (the direction of arrow X1) from the distal-end opening of the first shaft 24. A proximal portion of the second shaft 48 protrudes in a proximal direction (the direction of the arrow X2) from the proximal-end opening of the first shaft 24 (see FIG. 2). The second shaft 48 is provided in such a way as to extend along the first shaft 24 and be movable along the first shaft 24. The second shaft 48 is not limited to a tubular body, and may be a body other than a tubular body, instead.

A resilient member such as a spring may be provided between the first lumen 28 of the first shaft 24 and the second shaft 48, so that the second shaft 48 is configured to be biased in the proximal direction (the direction of the arrow X2) with respect to the first shaft 24 by a resilient force of the resilient member. As a result, when the user moves the second shaft 48 in the distal direction (the direction of the arrow X1) with respect to the first shaft 24, operation force of the user disappears, so that the second shaft 48 can move in the proximal direction by the resilient force.

The second shaft 48 is configured to follow the shape of the first expansion body 26. As a constituent material from which the second shaft 48 may be made, for example, a material more flexible than the constituent material of the first shaft 24 is selected. Specifically, examples of the constituent material of the second shaft 48 include a polyamide elastomer, a polyester elastomer, a polyurethane elastomer, polyvinyl chloride, polybutadiene, a silicone rubber, and a metal coil (including a composite with a resin). The second shaft 48 is flexible.

As illustrated in FIG. 5, the second shaft 48 includes a carrier holding portion 54 and a pressure-application portion 56 that is the distal portion of the second shaft 48. The pressure-application portion 56 includes an elastic body such as an elastomer member. The pressure-application portion 56 presses the first expansion body 26 against an inner surface of the outer cylinder 22 with the first expansion body 26 retracted in the outer cylinder 22.

A distal end of the carrier holding portion 54 includes a pressing surface 58. The carrier holding portion 54 can cause the pressing surface 58 to press an outer edge surface of the medical sheet 300 supported by the first expansion body 26 in the distal direction (the direction of the arrow X1). In the present embodiment, the pressure-application portion 56 is provided with the carrier holding portion 54 that supports the second expansion body 50. The carrier holding portion 54 includes the pressing surface 58 and an attachment hole 60. The carrier holding portion 54 is formed in such a way as to become wider toward the distal end when viewed from above. The carrier holding portion 54 is formed in a trapezoidal shape when viewed from above. In the cross section orthogonal to the axis of the second shaft 48 illustrated in FIG. 6, the carrier holding portion 54 has a line-symmetrical shape with respect to an imaginary line L1 passing through a center line C of the carrier holding portion 54 and parallel to the width direction of the carrier holding portion 54. In other words, the carrier holding portion 54 has a symmetrical shape in a vertical direction with respect to the imaginary line L1.

Specifically, in the cross section orthogonal to the axis of the second shaft 48, a cross section of the carrier holding portion 54 includes a pair of linear portions 621 and 622 orthogonal to the axis of the second shaft 48 and a pair of convex portions 641 and 642 disposed on both sides of the pair of linear portions 621 and 622 and convex in a direction (outward in the width direction) away from the linear portions 621 and 622. The convex portions 641 and 642 are formed in an arc shape in cross section. A first length D1 (width dimension) of the carrier holding portion 54 along an extending direction of the linear portions 621 and 622 is larger than a second length D2 (thickness dimension) of the carrier holding portion 54 orthogonal to the extending direction. That is, the carrier holding portion 54 has a flattened cross-sectional shape. The carrier holding portion 54 is not limited to a configuration formed in a flattened cross-sectional shape, and may be formed in, for example, a substantially circular cross-sectional shape, a substantially elliptical cross-sectional shape, a substantially square cross-sectional shape, a substantially rectangular cross-sectional shape, or the like, instead. In addition, instead of the convex portions 641 and 642, the carrier holding portion 54 may have an uneven structure in which the first expansion body 26 is pressed against the inner surface of the outer cylinder 22 in a state where the first expansion body 26 is housed in the outer cylinder 22.

When the first expansion body 26 is housed in a lumen 78 of the outer cylinder 22 together with the pressure-application portion 56, a width (first length D1) of the pressure-application portion 56 is set such that the first expansion body 26 is pressed toward the lumen 78 of the outer cylinder 22 by both ends (the pair of convex portions 641 and 642) of the pressure-application portion 56 in the width direction. The first length D1 of the pressure-application portion 56 is set, for example, to be equal to or slightly larger than an inner diameter of the outer cylinder 22 (a diameter of the lumen 78). Since the first expansion body 26 has a thickness, the first length D1 of the pressure-application portion 56 may be set slightly smaller than the inner diameter of the outer cylinder 22.

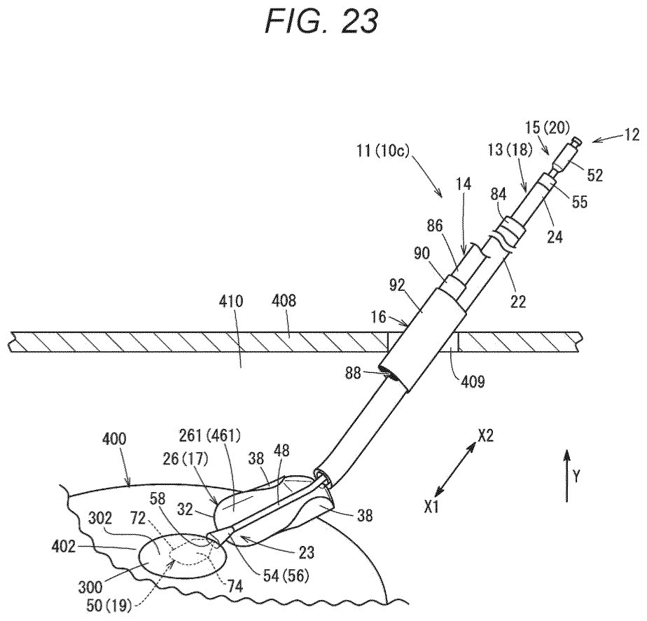

In FIG. 5, the pressing surface 58 is provided on a distal end surface of the carrier holding portion 54. The attachment hole 60 opens in the pressing surface 58. The second expansion body 50 is attached to the pressing surface 58. The pressing surface 58 presses the outer edge surface of the medical sheet 300 in the distal direction (the direction of the arrow X1) (see FIG. 23). The pressing surface 58 is a flat surface orthogonal to the axis of the carrier holding portion 54. The pressing surface 58 may include a supply hole (not illustrated) through which a liquid (for example, physiological saline solution) can be supplied toward the distal portion of the second shaft 48 via the second lumen 57 of the second shaft 48. The liquid is, for example, for priming or for wetting.

The attachment hole 60 opens in the pressing surface 58 of the carrier holding portion 54. The attachment hole 60 is disposed in the pressing surface 58 on the center line C of the carrier holding portion 54. The attachment hole 60 has a slit shape extending parallel to the width direction of the carrier holding portion 54 from the center line C. The attachment hole 60 has a symmetrical shape in the width direction with respect to the center line C. The attachment hole 60 extends in the axial direction from the pressing surface 58. A part of the second expansion body 50 is inserted into and connected to the attachment hole 60.

As illustrated in FIGS. 3 to 5, in the present embodiment, the second expansion body 50 is the sheet-like second support portion 19 having flexibility. Therefore, the second inner member 15 is the second carrier member 20 including the second shaft 48 and the second support portion 19. The second expansion body 50 includes a second joint 70 and a second support body 72. The second joint 70 is provided at a proximal end of the second expansion body 50. The second joint 70 is provided at a proximal end of the second support body 72. The second joint 70 is inserted into and, for example, bonded to the attachment hole 60 of the carrier holding portion 54. The second joint 70 may be joined to the attachment hole 60 of the carrier holding portion 54 by a suitable joining method other than bonding. Furthermore, the second expansion body 50 may be integrally formed with the carrier holding portion 54.

The second support body 72 extends in the distal direction (the direction of the arrow X1) from the second joint 70. The second support body 72 extending from the second joint 70 is shorter than the first support body 32 extending from the first joint 30. The second support body 72 is provided with, on an upper surface thereof, a second support surface 74 on which the medical sheet 300 is placed. The second support surface 74 is a flat surface. The second support body 72 is smaller than the first support body 32. That is, the second support surface 74 is smaller in area than the first support surface 261.

As illustrated in FIG. 4, a proximal portion of the second support body 72 is formed in such a way as to be narrower toward the proximal end (the direction of the arrow X2). An intermediate portion between a distal end and the proximal end of the second support body 72 extends with a substantially constant width. The distal portion of the second expansion body 50 protrudes in an arc shape in the distal direction (the direction of the arrow X1). A lubricant may be applied to both surfaces (a lower surface and an upper surface) of the second support body 72.

In FIG. 3, the hub 52 is attached to the proximal portion of the second shaft 48.

In FIGS. 2 and 3, the outer cylinder 22 is a cylindrical member having the lumen 78. The lumen 78 has a distal-end opening 80 that opens at a distal end (an end in the direction of the arrow X1) of the outer cylinder 22. The lumen 78 opens at a proximal end (an end in the direction of arrow X2) of the outer cylinder 22. The outer cylinder 22 is flexible. Examples of a constituent material from which the outer cylinder 22 may be made are the same as the examples of the constituent material of the first shaft 24 described above. When the first shaft 24 includes a flexible tube portion, the outer cylinder 22 can bend the first shaft 24. Furthermore, in the present embodiment, the outer cylinder 22 may include the flexible tube portion described above.

The first shaft 24 is inserted into (positioned inside) the lumen 78 of the outer cylinder 22. The outer cylinder 22 is shorter than the first shaft 24 in the axial direction. In FIGS. 4 and 5, the inner diameter of the outer cylinder 22 is smaller than the width of the intermediate support portion 36. The width of the intermediate support portion 36 is substantially equal to or smaller than a circumferential length of the inner surface of the outer cylinder 22 in order to allow the first expansion body 26 to be housed in the outer cylinder 22 with the first expansion body 26 curled into a cylinder along a circumferential direction of an inner peripheral surface of the outer cylinder 22. The outer cylinder 22 is provided with, at the proximal end thereof, an airtight valve 84 that is in close contact with an outer peripheral surface of the first shaft 24.

In FIGS. 3 and 5, a distal end surface of the outer cylinder 22 extends orthogonally to the axial direction of the outer cylinder 22.

As illustrated in FIG. 3, the endoscope 14 includes an elongated endoscope body 86. A distal portion of the endoscope body 86 is fixed to the outer peripheral surface of the outer cylinder 22 by the fixing member 16 (see FIG. 2). An objective lens 88 provided on a distal end surface of the endoscope body 86 is oriented toward the distal end of the outer cylinder 22 (the direction of the arrow X1). A distal portion of the endoscope body 86 is fixed to an intermediate portion of the outer cylinder 22 in the axial direction. The distal portion of the endoscope body 86, however, may be fixed to a distal portion of the outer cylinder 22.

The fixing member 16 includes, for example, a fixed cylinder 90 and a fixing tube 92. The fixed cylinder 90 is composed of, for example, a hard resin material. The endoscope body 86 can be inserted into a lumen of the fixed cylinder 90. The fixed cylinder 90 is disposed along a longitudinal direction of the outer cylinder 22. The fixing tube 92 is a tube for fixing the fixed cylinder 90 at a predetermined position on the outer cylinder 22. The fixing tube 92 is, for example, a heat-shrink tube. The outer cylinder 22 and the fixed cylinder 90 may be integrally molded. A method for fixing the distal portion of the endoscope body 86 to the outer cylinder 22, however, may be determined as desired.

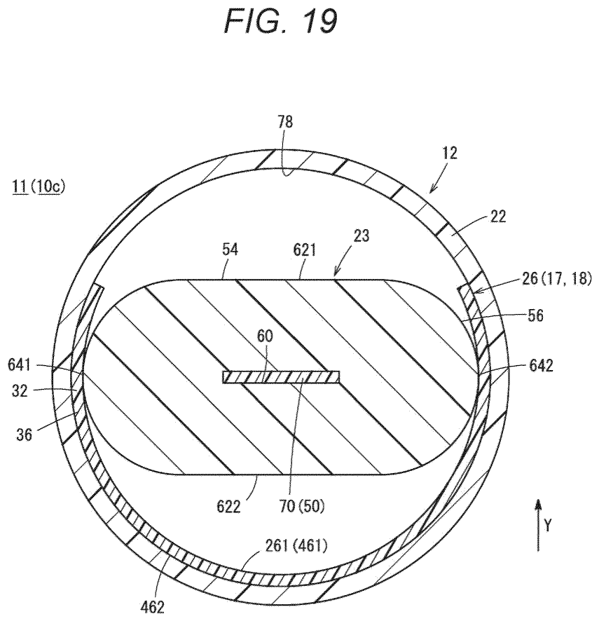

The movement restriction portion 23 can restrict relative movement of the first shaft 24 and the second shaft 48 in the axial direction. The movement restriction portion 23 is configured by the carrier holding portion 54 (pressure-application portion 56) configured by the distal portion of the second shaft 48. With the first expansion body 26 housed in the outer cylinder 22, the carrier holding portion 54 presses the first expansion body 26 against the inner surface (lumen 78) of the outer cylinder 22, so that relative displacement of the first expansion body 26 and the second shaft 48 is restricted. As the relative movement of the first expansion body 26 and the second shaft 48 is restricted, the relative movement of the first shaft 24 and the second shaft 48 in the axial direction is restricted (see FIG. 19).

The first and second expansion bodies 26 and 50 are not limited to flexible sheet shapes capable of holding the medical sheet 300. Any configuration may be employed as long as the first and second expansion bodies 26 and 50 can be extended (expanded) by protruding (being exposed) in the distal direction from the distal-end opening 80 of the outer cylinder 22. When each medical device 10c is not the transfer instrument 11, other modes of the first and second expansion bodies 26 and 50 include, for example, stents, network structures composed of a metal material or a resin material, and the like.

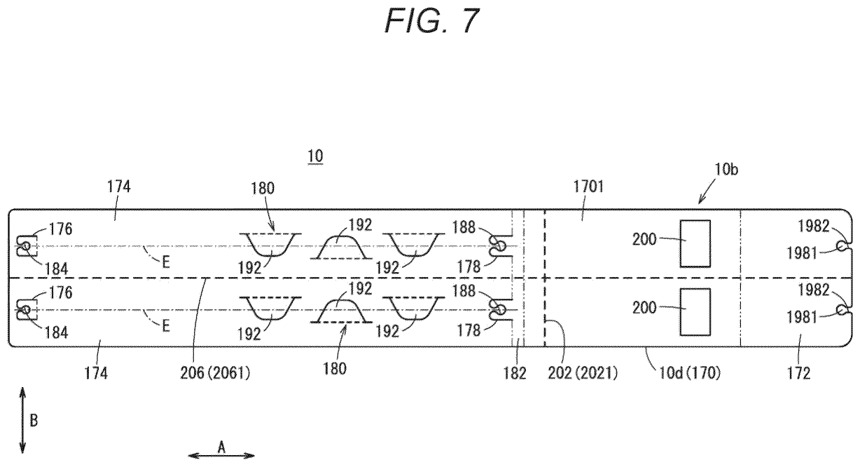

As illustrated in FIG. 7, the mount 10d of the medical device holding structure 10b is formed of, for example, thick paper and is formed in a rectangular shape or an elongated shape in plan view. An elongated direction of the mount 10d will be referred to as a longitudinal direction (a direction of an arrow A) hereinafter. A lateral direction of the mount 10d orthogonal to the longitudinal direction will be referred to as a width direction (a direction of an arrow B).

As illustrated in FIG. 1, the mount 10d includes a mount body 170 and a cover portion 172 provided at a distal end of the mount body 170 and continuous with the mount body 170. The mount 10d includes a plurality of device holding portions 174. The plurality of device holding portions 174 is arranged in parallel in the width direction (the direction of the arrow B) of the mount 10d. Each of the plurality of device holding portions 174 is capable of holding the medical device 10c. A case where two device holding portions 174 are provided for the mount 10d will be described hereinafter. The number of the device holding portions 174 is not limited to two, and may be one, or two or more.

The mount body 170 extends in the longitudinal direction (the direction of the arrow A) of the mount 10d. An upper surface of the mount body 170 includes a holding surface 1701 capable of holding the medical devices 10c. The mount body 170 includes first and second support pieces 176 and 178, claw portions 180, and a stepped portion 182. The first and second support pieces 176 and 178 and the claw portion 180 are provided in each device holding portion 174.

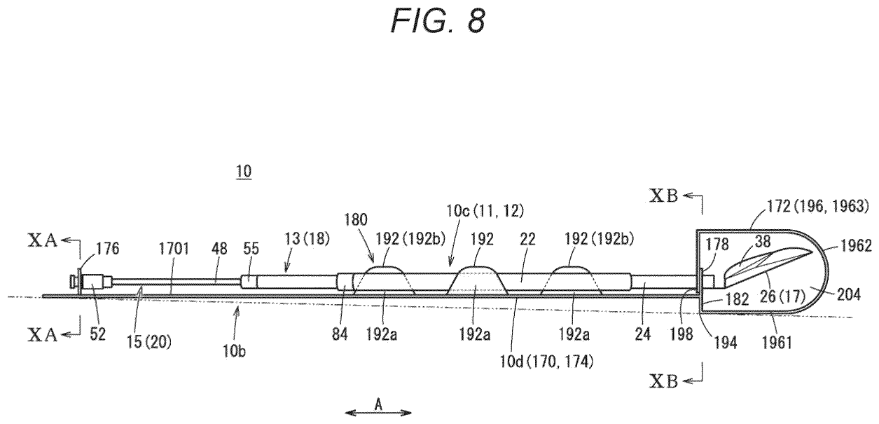

The first support piece 176 can hold the hub 52 of the medical device 10c. The first support piece 176 is provided in the vicinity of a proximal end of the mount body 170 in the longitudinal direction. The first support piece 176 is formed in a substantially rectangular shape and is formed by cutting out a part of the mount body 170. With respect to the flat mount body 170 illustrated in FIG. 7, the first support piece 176 is cut and raised from the mount body 170 and is substantially orthogonal to the holding surface 1701 of the mount body 170 (see FIG. 8). At this time, a lower end of the first support piece 176 is a fixed end fixed to the mount body 170, and an upper end of the first support piece 176 is a free end separated from the mount body 170. As illustrated in FIG. 8, the first support piece 176 protrudes upward from the holding surface 1701 of the mount body 170. In other words, the first support piece 176 stands upright by being bent with respect to the mount body 170. The first support pieces 176 of the device holding portions 174 are arranged at the same position in the longitudinal direction (the direction of the arrow A) of the mount body 170.

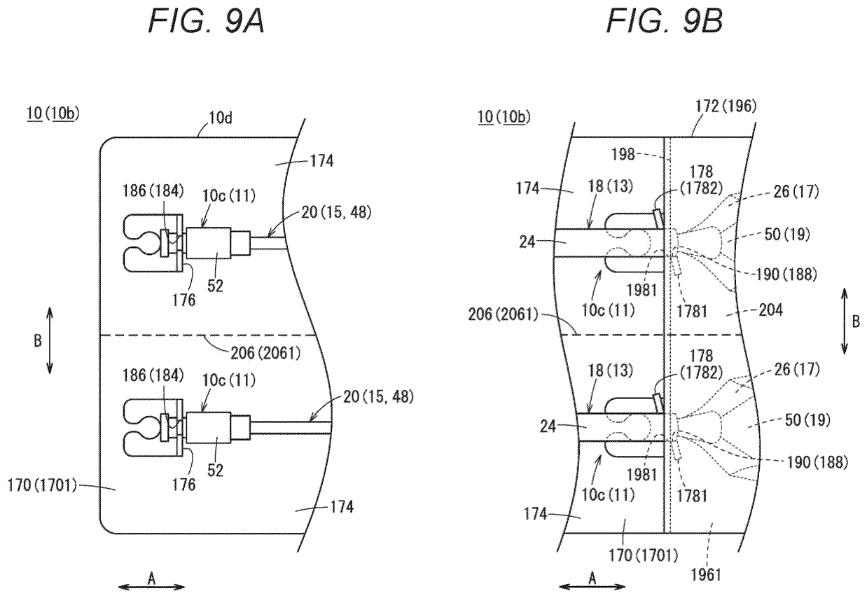

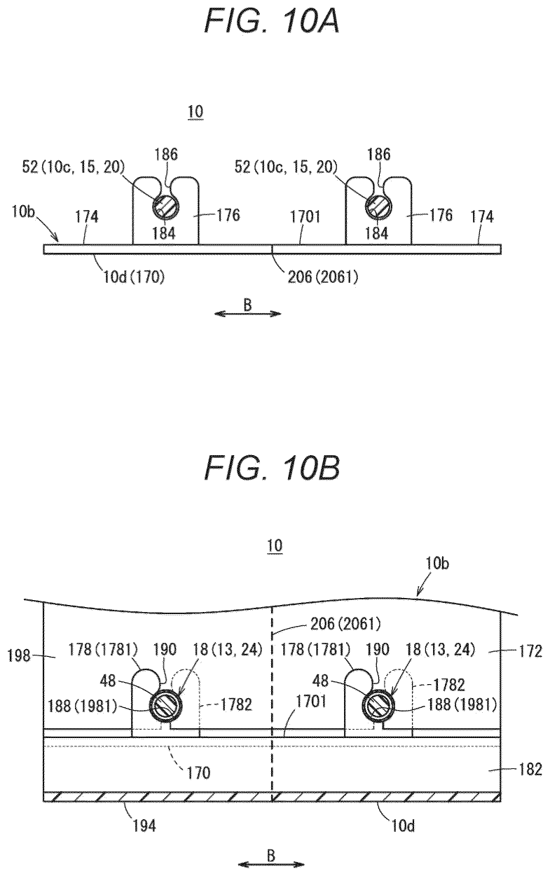

As illustrated in FIG. 9A, each first support piece 176 has a first support hole 184 into which the hub 52 of the medical device 10c is inserted and held. The first support hole 184 penetrates the first support piece 176 in a thickness direction (the longitudinal direction of the mount body 170). As illustrated in FIG. 10A, the first support hole 184 has a substantially circular cross section into which the hub 52 can be inserted. A diameter of the first support hole 184 corresponds to a diameter of the hub 52. The first support hole 184 opens to an upper portion of the first support piece 176 through a first insertion port 186. By inserting the hub 52 of the medical device 10c into the first support hole 184 through the first insertion port 186, an outer peripheral surface of the hub 52 is held by the first support piece 176. At this time, the holding surface 1701 of the mount body 170 and the hub 52 are separated from each other.

The second support piece 178 can hold the vicinity of the distal end of the first shaft 24 of the medical device 10c. The second support piece 178 is provided in the vicinity of a distal end of the mount body 170 in the longitudinal direction. The second support piece 178 is formed in a substantially rectangular shape and is formed by cutting out a part of the mount body 170. The second support piece 178 is cut and raised with respect to the flat mount body 170 illustrated in FIG. 7, and is substantially orthogonal to the holding surface 1701 of the mount body 170 (see FIG. 8). At this time, a lower end of the second support piece 178 is a fixed end fixed to the mount body 170, and an upper end of the second support piece 178 is a free end separated from the mount body 170. As illustrated in FIG. 8, the second support piece 178 protrudes upward from the holding surface 1701 of the mount body 170. In other words, the second support piece 178 stands upright by being bent with respect to the mount body 170. The second support pieces 178 of the device holding portions 174 are arranged at the same position in the longitudinal direction (the direction of the arrow A) of the mount body 170. In the mount body 170, the first support piece 176 and the second support piece 178 face each other in the longitudinal direction (see FIG. 1). The first support piece 176 and the second support piece 178 are separated from each other in the longitudinal direction of the mount body 170 and are parallel to each other.

As illustrated in FIG. 9B, the second support piece 178 has a second support hole 188 into which the first shaft 24 is inserted and held. The second support hole 188 penetrates the second support piece 178 in a thickness direction (the longitudinal direction of the mount body 170). As illustrated in FIG. 10B, the second support hole 188 has a substantially circular cross section into which the first shaft 24 can be inserted. A diameter of the second support hole 188 corresponds to a diameter of the first shaft 24. The second support hole 188 opens to an upper portion of the second support piece 178 through a second insertion port 190. By inserting the first shaft 24 of the medical device 10c into the second support hole 188 through the second insertion port 190, the outer peripheral surface of the first shaft 24 is held by the second support piece 178. At this time, the holding surface 1701 of the mount body 170 and the first shaft 24 are separated from each other.

As illustrated in FIG. 10B, the second support piece 178 has a pair of engagement ends 1781 and 1782 arranged on both sides of the second insertion port 190 and the second support hole 188. The engagement ends 1781 and 1782 face each other with the second insertion port 190 and the second support hole 188 interposed therebetween.

As illustrated in FIG. 8, when the medical device 10c is held in the first support hole 184 of the first support piece 176 and the second support hole 188 of the second support piece 178, the medical device 10c and the mount body 170 are substantially parallel to each other.

As illustrated in FIG. 1, each claw portion 180 is disposed between the first support piece 176 and the second support piece 178 in the mount body 170. The claw portion 180 includes a plurality of hooks 192. The hooks 192 are cut and raised from the mount body 170 illustrated in FIG. 7 with respect to the flat mount body 170. The plurality of hooks 192 is arranged in such a way as to alternate in the width direction with respect to a center line E of the device holding portion 174 connecting the center of the first support hole 184 of the first support piece 176 and the center of the second support hole 188 of the second support piece 178. A case where the claw portion 180 includes three hooks 192 will be described hereinafter.

The plurality of hooks 192 is alternately arranged in each device holding portion 174 on one side in the width direction and another side in the width direction with respect to the center line E in the longitudinal direction of the mount body 170. The plurality of hooks 192 is arranged at equal intervals in the longitudinal direction of the mount body 170. Each hook 192 includes a hook proximal portion 192a connected to the mount body 170 and a hook distal portion 192b arranged on a center line E side of the device holding portion 174 with respect to the hook proximal portion 192a. The hook proximal portion 192a is a fixed end fixed to the mount body 170. The hook distal portion 192b is a free end movable away from the mount body 170. The hook distal portion 192b is disposed in such a way as to cross the center line E of the device holding portion 174. The hook proximal portion 192a has a perforation, and the hook 192 is fixed to the mount body 170 through the perforation. The hook 192 can be easily cut and raised with respect to the mount body 170 thanks to the perforation.

When the medical device 10c is held by the mount body 170, the hook distal portion 192b of each hook 192 is arranged above the mount body 170 and is arranged above the medical device 10c to come into contact with the outer peripheral surface of the outer cylinder 22 of the medical device 10c (see FIG. 8). At this time, the plurality of hooks 192 alternately comes into contact with the outer peripheral surface of the outer cylinder 22 from the width direction, and holds the medical device 10c including the outer cylinder 22 between the holding surface 1701 of the mount body 170 and the hooks 192. The outer cylinder 22 of the medical device 10c is pushed toward the holding surface 1701 by the plurality of hooks 192. As a result, the medical device 10c is held by the plurality of hooks 192 between the first support piece 176 and the second support piece 178.

The stepped portion 182 is disposed on a distal end side of the mount body 170. The stepped portion 182 is disposed at a boundary between the mount body 170 and the cover portion 172. The stepped portion 182 is bent downward with respect to a distal end of the mount body 170. The stepped portion 182 extends downward from the distal end of the mount body 170. An upper end of the stepped portion 182 is connected to the distal end of the mount body 170. The second support pieces 178 are disposed at the upper end of the stepped portion 182. That is, the stepped portion 182 and the second support pieces 178 are arranged at the same position in the longitudinal direction of the mount 10d.

When the medical device package 10 is placed on a flat surface, a lower end of the stepped portion 182 serves as a base portion 194 with respect to the surface. The medical device package 10 is stably placed by bringing the base portion 194, which is the lower end of the stepped portion 182, and the proximal end of the mount body 170 into contact with the flat surface. At this time, the medical device package 10 is slightly inclined upward from the proximal end toward the distal end. The medical device package disclosed here is not limited to the case where the stepped portion 182 is provided. The mount body 170 and the cover portion 172 may be formed over the same surface along the longitudinal direction.

As illustrated in FIG. 8, the cover portion 172 is disposed in the distal direction of the mount body 170. The cover portion 172 is connected to the lower end (base portion 194) of the stepped portion 182. When the medical device 10c is held on the mount 10d, the cover portion 172 has a bent shape folded back in such a way as to cover the first and second expansion bodies 26 and 50 of the medical device 10c.

The cover portion 172 includes a cover body 196 and an engagement portion 198 engaged with the medical device 10c. The cover body 196 is connected to the lower end of the stepped portion 182. The cover body 196 extends in a direction away from the mount body 170 (a right direction in FIG. 8), and is then bent upward and folded back toward the proximal side of the mount body 170 (a left direction in FIG. 8). The cover body 196 includes a bottom portion 1961 extending flat from the stepped portion 182, a curved portion 1962 having an arcuate cross section curved upward and folded back from an end of the bottom portion 1961 on a side opposite to the stepped portion 182, and a ceiling portion 1963 extending from an upper end of the curved portion 1962 toward the first support piece 176. The bottom portion 1961 and the ceiling portion 1963 are separated from each other in the vertical direction and are substantially parallel to each other. When viewed from the width direction of the mount 10d illustrated in FIG. 8, the cover body 196 has a substantially U-shape folded back via the curved portion 1962.

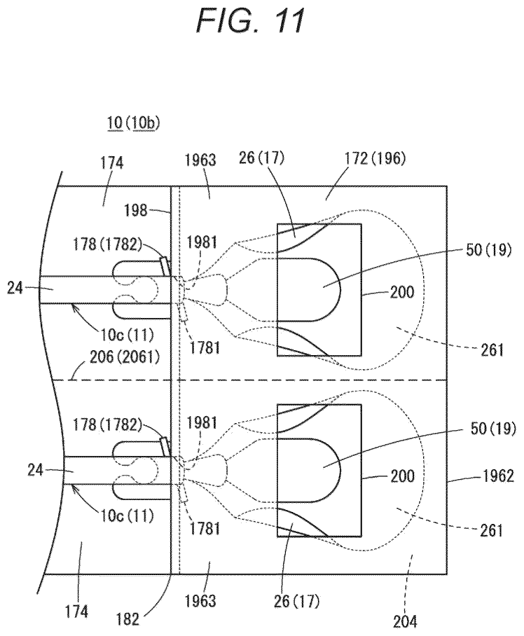

As illustrated in FIG. 11, each ceiling portion 1963 has an opening 200 for visual recognition. The opening 200 is disposed in each device holding portion 174. The openings 200 are opened in parallel in the width direction of the curved portions 1962. The user can visually recognize an inside of the cover portion 172 from the outside of the ceiling portions 1963 through the openings 200. When the mount body 170 is formed of an opaque material, a configuration may be achieved in which the inside of the cover portion 172 is visible from the outside by making portions corresponding to the openings 200 transparent.

As illustrated in FIG. 8, the engagement portion 198 is bent downward with respect to the ceiling portion 1963 and extends downward.

As illustrated in FIG. 9B, a lower end of the engagement portion 198 has an engagement hole 1981 engageable with the first shaft 24 of the medical device 10c. The engagement hole 1981 penetrates the engagement portion 198 in the thickness direction (the longitudinal direction of the mount body 170). The engagement hole 1981 has a substantially circular cross section into which the first shaft 24 can be inserted (see FIG. 10B). A diameter of the engagement hole 1981 corresponds to the diameter of the first shaft 24. The engagement hole 1981 has substantially the same shape as the second support hole 188 of the second support piece 178. The engagement hole 1981 opens to a lower portion of the engagement portion 198 through the engagement port 1982 (see FIG. 7). By inserting the first shaft 24 of the medical device 10c into the engagement hole 1981 through the engagement port 1982, the outer peripheral surface of the first shaft 24 and the engagement portion 198 are engaged with each other.

In a plan view of the medical device holding structure 10b illustrated in FIG. 9B, the second support piece 178 and the engagement portion 198 are engaged with each other via the engagement hole 1981 and the second support hole 188 in such a way as to cross each other. More specifically, the engagement end 1781 of the second support piece 178 is disposed on a housing portion 204 side with respect to the engagement portion 198, and the engagement end 1782 is disposed on a first support piece 176 side with respect to the engagement portion 198. That is, the second support piece 178 is slightly inclined with respect to the engagement portion 198 extending in the width direction.

The engagement portion 198 is held by the first shaft 24 by engaging the engagement hole 1981 of the engagement portion 198 with the first shaft 24 in a state where the medical device 10c is held by the first and second support pieces 176 and 178 illustrated in FIG. 8. When the engagement portion 198 is held by the first shaft 24, the distal portion (first and second expansion bodies 26 and 50) of the medical device 10c is covered by the cover portion 172. That is, the first shaft 24 of the medical device 10c is supported by the second support piece 178, and the engagement portion 198 of the cover portion 172 is held by the first shaft 24. As a result, the housing portion 204 surrounded by the cover body 196 and the engagement portion 198 is formed inside the cover portion 172. The distal portion of the medical device 10c can be housed in the housing portion 204. The housing portion 204 is opened on both sides in the width direction of the cover portion 172.

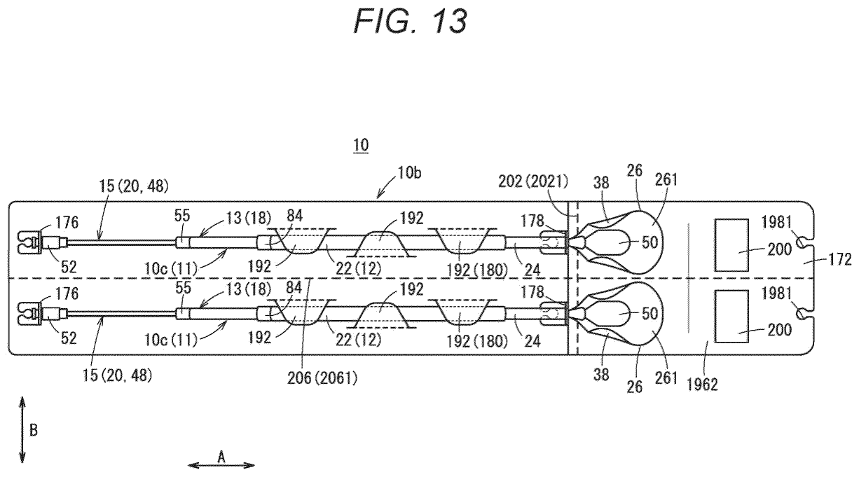

As illustrated in FIG. 9B, the bottom portion 1961 of the cover body 196 includes a cover separation portion 202 (see FIG. 13). The cover separation portion 202 can be used to separate the cover portion 172 from the mount body 170, and is disposed, for example, away from the stepped portion 182. The cover separation portion 202 extends linearly along the width direction (the direction of the arrow B) of the mount 10d. The cover separation portion 202 is a first break line 2021 that can be used to separate the bottom portion 1961 of the cover portion 172 in the longitudinal direction of the mount 10d. The first break line 2021 is a perforation having a plurality of notches cut out in the thickness direction of the mount body 170. When the medical device 10c is taken out from the mount 10d, the first break line 2021 can be broken in a state where the engagement portion 198 is detached from the first shaft 24 and the cover portion 172 is unfolded.

As illustrated in FIG. 1, the mount 10d has a separation portion 206 at the center in the width direction of the mount body 170. The separation portion 206 is disposed at the center of the mount body 170 in the width direction. In other words, the separation portion 206 is disposed between the two device holding portions 174. The separation portion 206 extends linearly along the longitudinal direction (the direction of the arrow A) of the mount body 170. The separation portion 206 is a second break line 2061 that can be used to separate the mount body 170. The second break line 2061 is a perforation having a plurality of notches cut out in the thickness direction of the mount body 170. By breaking the second break line 2061, the mount body 170 can be separated in the width direction, and the two device holding portions 174 holding the medical devices 10c can be separated in the width direction. The separation portion 206 need not be provided in the mount body 170.

Next, a case where the medical devices 10c are held on the mount 10d to form the medical device holding structure 10b will be described.

First, a flat mount 10d (a mount 10d before deformation) illustrated in FIG. 7 is prepared, and the first and second support pieces 176 and 178 are raised from the mount body 170 to be upright, and the stepped portion 182 is bent with respect to the mount body 170. As a result, the first and second support pieces 176 and 178 are formed on the holding surface 1701 of the mount body 170, and the stepped portion 182 is formed between the mount body 170 and the cover portion 172.

The first and second expansion bodies 26 and 50 protrude outward from the distal-end opening 80 of the outer cylinder 22 in the medical device 10c, and the hub 52 of the medical device 10c is inserted into the first support hole 184 through the first insertion port 186. The outer peripheral surface of the hub 52 is held inside the first support hole 184. The first shaft 24 of the medical device 10c is inserted into the second support hole 188 through the second insertion port 190. The outer peripheral surface of the first shaft 24 is held in the second support hole 188 of the second support piece 178. The vicinity of the distal end of the first shaft 24 is held by the second support piece 178.

As a result, as illustrated in FIG. 8, the outer cylinder 22, the first inner member 13, and the second inner member 15 face the holding surface 1701 of the mount body 170 and are held substantially parallel to and separated from the holding surface 1701. The axis of the outer cylinder 22 and the first and second support pieces 176 and 178 are substantially orthogonal to each other. The plurality of hooks 192 is raised upward from the mount body 170, and the medical device 10c is disposed between the hook distal portions 192b of the hooks 192 and the mount body 170. The outer peripheral surface of the outer cylinder 22 is pushed and held toward the mount body 170 by the plurality of hooks 192. At this time, the first and second expansion bodies 26 and 50 face the bottom portion 1961 of the cover portion 172 and are disposed above the bottom portion 1961.

In the cover portion 172, after the engagement portion 198 is bent at a predetermined position with respect to the cover body 196, the cover body 196 is bent in such a way as to be folded back toward the outer cylinder 22. As a result, the cover body 196 of the cover portion 172 is curved in a substantially U-shape opened toward the outer cylinder 22. The engagement hole 1981 of the engagement portion 198 is moved from above the second support piece 178 toward the first shaft 24. The engagement hole 1981 is inserted into the first shaft 24 from above the first shaft 24 through the engagement port 1982. The engagement portion 198 is held by the first shaft 24. The engagement portion 198 is disposed along a vertical direction orthogonal to the axis of the first shaft 24. As a result, the first and second expansion bodies 26 and 50 are covered with the bottom portion 1961, the curved portion 1962, and the ceiling portion 1963 of the cover body 196 and the engagement portion 198.

More specifically, the first and second expansion bodies 26 and 50 are covered by the cover portion 172 in the longitudinal direction and the vertical direction of the mount 10d and housed in the housing portion 204. At this time, since the housing portion 204 has a predetermined volume in the longitudinal direction and the vertical direction of the mount 10d, the first and second expansion bodies 26 and 50 and the cover portion 172 are disposed apart from each other and do not come into contact with each other. The user can visually recognize the first and second expansion bodies 26 and 50 from the outside of the cover portion 172 through the opening 200 (see FIG. 11).

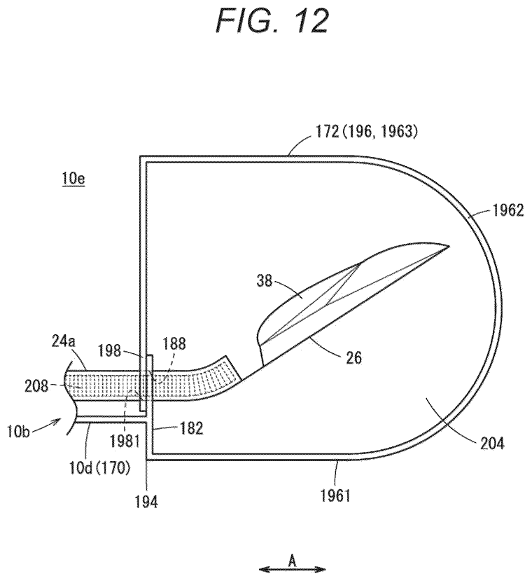

As illustrated in FIG. 12, a medical device 10e according to a modification can be held in the medical device holding structure 10b. A first shaft 24a of the medical device 10e has flexibility. The first shaft 24a includes therein a flexible tube portion 208 capable of maintaining a bent shape. In the medical device 10e, the first shaft 24a can be bent into an appropriate shape, and the bent shape can be maintained. The first and second expansion bodies 26 and 50 can be housed in the housing portion 204 with a distal portion of the first shaft 24a bent.

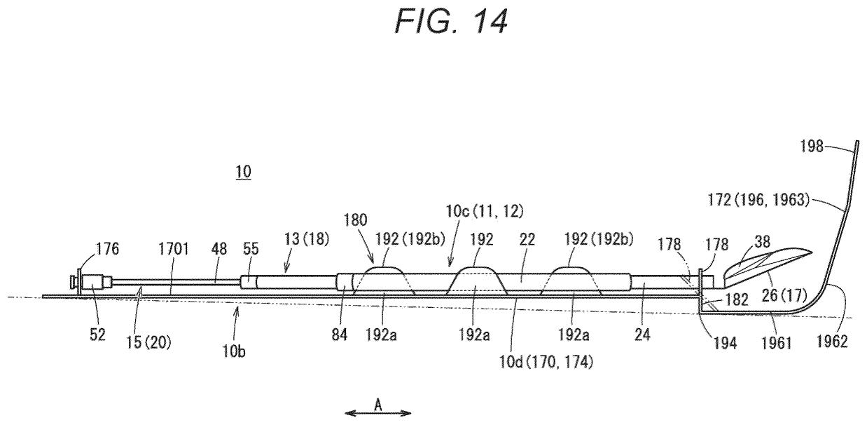

In FIG. 8, when the medical device 10c is taken out from the mount 10d, the engagement portion 198 of the cover portion 172 is moved upward in such a way as to be separated from the medical device 10c. The engagement of the engagement hole 1981 with the first shaft 24 is released. Furthermore, by opening the cover portion 172, as illustrated in FIGS. 13 and 14, the proximal direction and an upper side of the housing portion 204 are opened, and the cover portion 172 is in an unfolded state.

When the cover portion 172 is unfolded in a direction away from the first and second expansion bodies 26 and 50, the second support piece 178 tilts toward the holding surface 1701 of the mount body 170 with the fixed end of the second support piece 178 as a fulcrum (see a dash-dot-dot line shape in FIG. 14). The user grips the medical device 10c and moves the medical device 10c upward with respect to the mount 10d in such a way as to separate the medical device 10c from the mount 10d. The holding of the medical device 10c by the mount 10d is released, and the medical device 10c is taken out from the mount 10d.

At this time, the user can remove the unfolded cover portion 172 from the mount 10d by separating the cover portion 172 in the distal direction using the cover separation portion 202. The unfolded cover portion 172 is prevented from being an obstacle when the medical device 10c is taken out.

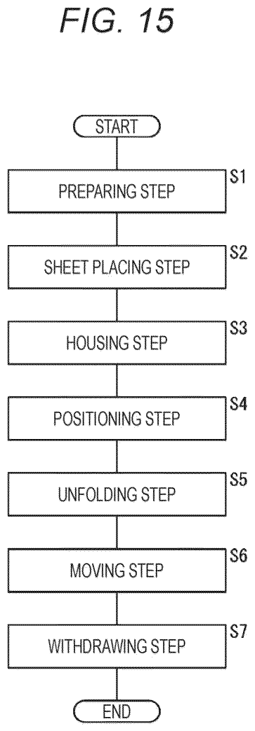

Next, a transfer method for transferring the medical sheet 300 to a treatment site of a living body will be described. Specifically, as illustrated in FIGS. 21 to 24, a transfer method for transferring the medical sheet 300 to the recipient site 402 of the heart 400 (the treatment site of the living body) during thoracoscopic surgery will be described. As illustrated in FIG. 15, the transfer method according to the present embodiment includes a preparing step, a sheet placing step, a housing step, a positioning step, an unfolding step, a moving step, and a withdrawing step.

First, in the preparing step (step S1), the transfer instrument 11 according to the present embodiment described above is prepared. The following description will be given on the assumption that the state illustrated in FIG. 2 is an initial state of the transfer instrument 11. In the initial state, the first and second shafts 24 and 48 are moved in the distal direction (the direction of the arrow X1) relative to the outer cylinder 22 in such a way as to achieve a protruding position (second position), where the first expansion body 26 and the second expansion body 50 protrude from the distal-end opening 80 of the outer cylinder 22 in the distal direction. The first and second support portions 26 and 50 are each exposed from the outer cylinder 22 in the distal direction and unfolded, and the second expansion body 50 is positioned on the first support surface 261 of the first expansion body 26. That is, the second expansion body 50 is positioned at a retracted position where the second expansion body 50 overlaps the first support surface 261 of the first expansion body 26. At this time, the proximal portion of the carrier holding portion 54 is in the first lumen 28 of the first shaft 24.

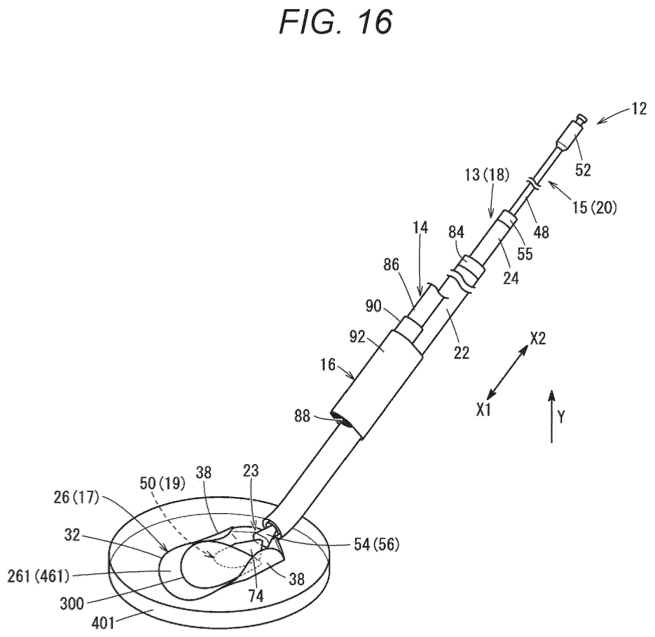

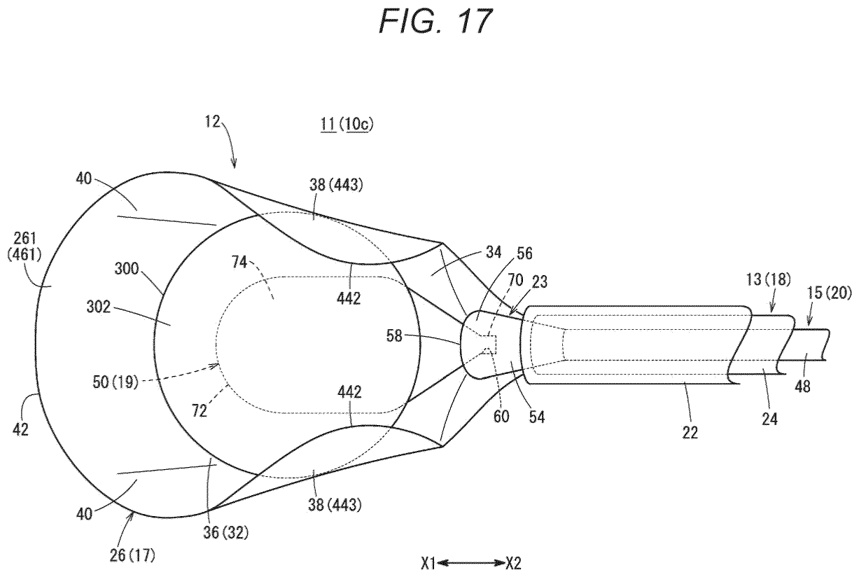

Next, in the sheet placing step (step S2), the medical sheet 300 placed on a Petri dish 401 is placed on the second support surface 74 as illustrated in FIG. 16. As illustrated in FIG. 17, the medical sheet 300 sticks out over the second expansion body 50 with the medical sheet 300 placed on the second support surface 74. The first support surface 261 supports an overhanging portion 302 of the medical sheet 300 that sticks out from the second expansion body 50. The pair of second protrusions 40 prevents the medical sheet 300 from moving (deviating) in the width direction of the intermediate support portion 36 with the medical sheet 300 placed on the second support surface 74.

Subsequently, in the housing step (step S3 in FIG. 15), the medical sheet 300 is housed in the outer cylinder 22 at a housed position (first position) together with the first expansion body 26 and the second expansion body 50. Specifically, the first shaft 24 of the first carrier member 18 and the second shaft 48 of the second carrier member 20 are moved together in the proximal direction (the direction of the arrow X2) relative to the outer cylinder 22.

Accordingly, the proximal-end support portion 34 is pulled in the proximal direction through the distal-end opening 80 of the outer cylinder 22. At this time, when both tapered sides of the proximal-end support portion 34 come into contact with the distal-end opening 80 of the outer cylinder 22, a force acts on the proximal-end support portion 34 to cause the proximal-end support portion 34 to curl along a circumferential direction of the outer cylinder 22. Therefore, the proximal-end support portion 34 is curled and smoothly pulled into the outer cylinder 22. At this time, the first expansion body 26 is housed in the outer cylinder 22 while curling in a conical shape such that the distal end side of the first expansion body 26 becomes larger than the proximal-end support portion 34 in diameter.

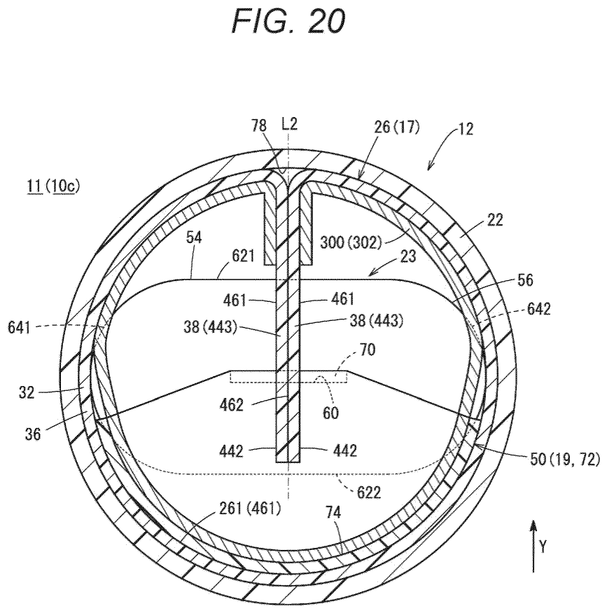

When the proximal-end support portion 34 is deformed, a force acts on the intermediate support portion 36 to cause the intermediate support portion 36 to curl along the circumferential direction of the outer cylinder 22, so that the intermediate support portion 36 is curled and pulled into the outer cylinder 22. At this time, the intermediate support portion 36 is deformed into a cylindrical shape along the inner surface of the outer cylinder 22. Each of the pair of first protrusions 38 is curved with the front surface 461 of the first expansion body 26 facing inward and the back surface 462 of the first expansion body 26 facing outward such that the fixed end 441 thereof is rolled inward. As illustrated in FIG. 20, the back surfaces 462 of the intermediate portions 443 extending, in a convex shape, outward in a radial direction come into contact with each other on an imaginary line L2 extending in a direction orthogonal to a center axis of the outer cylinder 22. The intermediate portion 443 of one of the first protrusions 38 and the intermediate portion 443 of the other first protrusion 38 come into contact with each other and are housed downward (the first support surface 261 and the front surface 461).

Accordingly, the back surface 462 of the first expansion body 26 is curved to come into close contact with the inner surface of the outer cylinder 22, each of the first protrusions 38 is further curved from the fixed end 441 toward the free end 442 to fold back toward the center of the outer cylinder 22, and the pair of free ends 442 is positioned below the center axis of the outer cylinder 22. That is, the first expansion body 26 is curved in a heart shape along the inner surface of the outer cylinder 22.

The heart shape refers to a substantially circular shape including two convex shapes: a convex shape on one side and another convex shape on the other side. When the heart shape is formed in the lumen 78 of the tubular body (outer cylinder 22), the two convex shapes protruding toward the opposite sides along the inner surface of the tubular body come close to each other so as to bring peripheral surfaces thereof into partial contact with each other, so that the entire contour becomes a substantially circular shape along the inner surface of the tubular body (see the shape of the first expansion body 26 in FIG. 20).

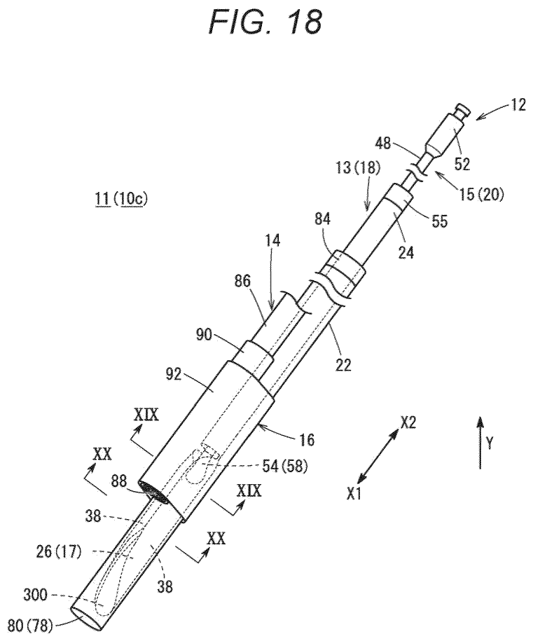

As the first expansion body 26 is curved and deformed, the second expansion body 50 is similarly curved and deformed along the first expansion body 26 inside the first expansion body 26 (on a surface 461 side). As the first and second expansion bodies 26 and 50 are curved and deformed, the medical sheet 300 is deformed into a shape corresponding to the shapes of the first and second support bodies 32 and 72, and the medical sheet 300 is housed in the outer cylinder 22. As illustrated in FIG. 18, the housing step is completed when the first expansion body 26 is entirely inserted into the outer cylinder 22.

In the housed state where the first expansion body 26, the second expansion body 50, and the medical sheet 300 are housed in the outer cylinder 22, the pair of first protrusions 38 is located, as illustrated in FIG. 20, adjacent to the distal end (the direction of the arrow X1) relative to the pressing surface 58 of the pressure-application portion 56 with the back surfaces 462 thereof in contact with each other (see FIG. 18).

Subsequently, in the positioning step (step S4 in FIG. 15), as illustrated in FIG. 21, the transfer instrument 11 is inserted into a chest cavity 410 through an incision 409 of a chest 408. At this time, the distal end of the transfer instrument 11 is positioned near the recipient site 402 of the heart 400, and the distal end of the endoscope 14 is positioned in the chest cavity 410. Before the transfer instrument 11 is inserted into the chest cavity 410, a liquid supply instrument (not illustrated) may be connected to a connection port of the hub 52 to introduce a liquid (for example, a physiological saline solution).

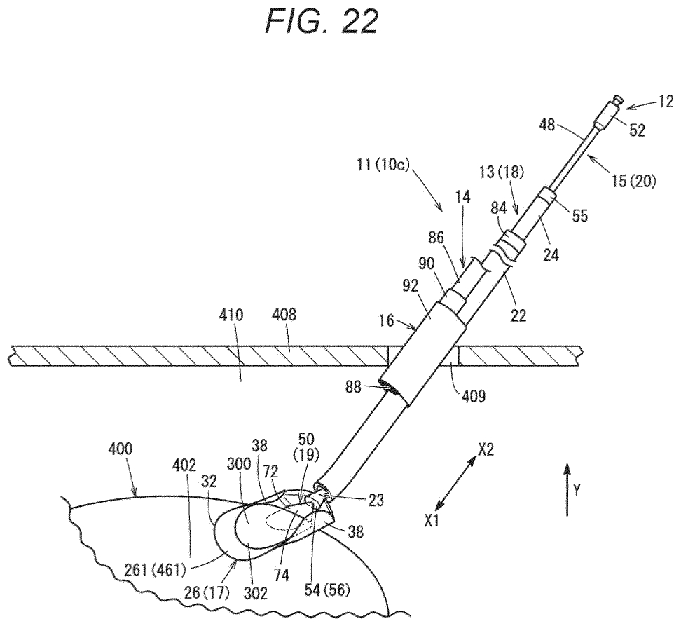

Next, in the unfolding step (step S5 in FIG. 15), as illustrated in FIG. 22, the first expansion body 26, the second expansion body 50, and the medical sheet 300 are unfolded. Specifically, in the unfolding step, the user grips the first shaft 24 and moves the first shaft 24 in the distal direction (the direction of the arrow X1) relative to the outer cylinder 22. As a result, the valve 55 of the first shaft 24 causes the second shaft 48 to move together with the first shaft 24 in the distal direction (the direction of the arrow X1). At this time, the first shaft 24 and the second shaft 48 integrally move in the distal direction via the movement restriction portion 23. The first expansion body 26 exposed from the distal-end opening 80 of the outer cylinder 22 then returns to an original shape due to restoring force thereof. At the second position where the first expansion body 26 is unfolded, the second expansion body 50 and the medical sheet 300 extend flat.

In the unfolding step, the second support surface 74 of the second carrier member 20 on which the medical sheet 300 is placed is entirely positioned on the first support surface 261. At this time, the medical sheet 300 is supported by the first support surface 261 and the second support surface 74. It is therefore possible to suppress occurrence of crinkles in the overhanging portion 302 of the medical sheet 300 before the medical sheet 300 is transferred to the recipient site 402 of the heart 400.

Next, in the moving step (step S6 in FIG. 15), as illustrated in FIG. 23, the second carrier member 20 is moved in the distal direction (the direction of the arrow X1) relative to the first carrier member 18, so that the second expansion body 50 on which the medical sheet 300 is placed is moved from the retracted position to an advanced position, and the second expansion body 50 protrudes in the distal direction (the direction of the arrow X1) relative to the distal end of the first expansion body 26. Specifically, in the moving step, the second shaft 48 is moved in the distal direction relative to the first shaft 24.

As a result, the second expansion body 50 moves in the distal direction (the direction of the arrow X1) relative to the first expansion body 26. At this time, when the pressing surface 58 of the carrier holding portion 54 (pressure-application portion 56) presses the outer edge surface of the medical sheet 300 in the distal direction, the medical sheet 300 is entirely positioned further in the distal direction than the first expansion body 26. In this moving step, the medical sheet 300 is moved to above the recipient site 402 of the heart 400 to bring the overhanging portion 302 of the medical sheet 300 into contact with the recipient site 402.

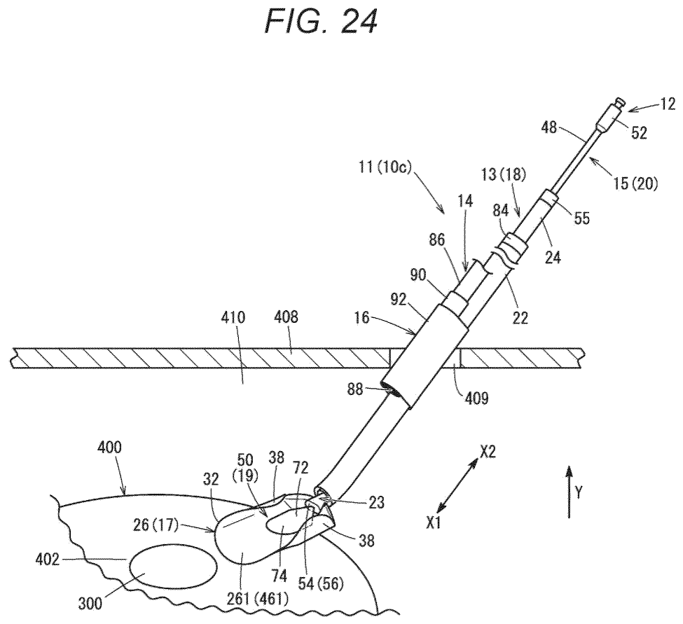

Subsequently, in the withdrawing step (step S7 in FIG. 15), as illustrated in FIG. 24, the second carrier member 20 is moved from the second position to the first position to withdraw the second expansion body 50 from between the recipient site 402 and the medical sheet 300. Accordingly, the medical sheet 300 entirely comes into contact with the surface of the recipient site 402. This is the end of the transfer of the medical sheet 300 to the recipient site 402. Subsequently, the transfer instrument 11 is withdrawn from the chest 408 with the first expansion body 26 and the second expansion body 50 housed in the outer cylinder 22.

The present embodiment has the following effects.

As illustrated in FIG. 8, in the medical device holding structure 10b, since the first and second expansion bodies 26 and 50 can be covered and protected by the cover portion 172, deformation of the first and second expansion bodies 26 and 50 due to an external force is prevented. As a result, the first and second expansion bodies 26 and 50 in the medical device holding structure 10b can be protected.

The mount 10d of the medical device holding structure 10b includes the second support piece 178 that is raised on the holding surface 1701 of the mount body 170 and that holds the medical device 10c, and when the cover portion 172 is unfolded in a direction of separating from the first and second expansion bodies 26 and 50, the second support piece 178 tilts toward the holding surface 1701. As a result, when the user unfolds the cover portion 172 and takes out the medical device 10c, the second support piece 178 tilts toward the holding surface 1701, so that the medical device 10c can be easily taken out from the mount 10d.

As illustrated in FIG. 1, since the mount 10d includes the plurality of device holding portions 174 capable of holding a plurality of medical devices 10c, it is possible to hold the plurality of medical devices 10c with the single mount 10d, which is efficient.

Between two adjacent device holding portions 174 among the plurality of device holding portions 174 in the mount 10d, the separation portion 206 that can be used to separate one device holding portion 174 and another device holding portion 174 is provided, and in a state where the medical devices 10c are held in the device holding portions 174, the mount 10d can be divided using the separation portion 206 for each medical device 10c to be used.

Since the mount 10d includes the cover separation portion 202 that can be used to separate the cover portion 172 from the mount body 170, when the medical device 10c is taken out from the mount 10d, the unfolded cover portion 172 can be separated and removed from the mount body 170 via the cover separation portion 202 including the first break line 2021. As a result, contact between the first and second expansion bodies 26 and 50 of the medical device 10c and the cover portion 172 is avoided, and the medical device 10c can be smoothly taken out.

As illustrated in FIG. 11, since the cover portion 172 has the openings 200 penetrating the cover portion 172, the first and second expansion bodies 26 and 50 can be visually recognized from the outside of the cover portion 172 in a state where the first and second expansion bodies 26 and 50 are housed in the cover portion 172.

As illustrated in FIG. 8, since the first and second expansion bodies 26 and 50 can be covered and protected by the cover portion 172 in the medical device holding structure 10b with the first and second expansion bodies 26 and 50 unfolded from the outer cylinder 22 of the medical device 10c, deformation due to long-term housing in the outer cylinder 22 is prevented from remaining in the first and second expansion bodies 26 and 50. As a result, while the first and second expansion bodies 26 and 50 are protected in the medical device holding structure 10b, the first and second expansion bodies 26 and 50 can be reliably and stably unfolded when the first and second expansion bodies 26 and 50 protrude from the outer cylinder 22 at the time of using the medical device 10c.

The first expansion body 26 is formed of the flexible sheet having the front surface 461 and the back surface 462. The first expansion body 26 includes the pair of first protrusions 38 protruding upward from both sides, in the width direction, of the first support surface 261 orthogonal to the moving direction of the first shaft 24. With this configuration, when the first expansion body 26 is housed in the outer cylinder 22, the back surfaces 462 of the pair of first protrusions 38 curved and deformed into a convex shape come into contact with each other. The pair of first protrusions 38 is moved toward the first support surface 261, and the first expansion body 26 is curved and deformed into a heart shape. Accordingly, since the first expansion body 26 can be housed in the outer cylinder 22 in the heart shape, it is possible to effectively reduce a possibility of breakage of the medical sheet 300 held by the first expansion body 26 as compared with a case where the first expansion body 26 is deformed into a shape other than the heart shape. Furthermore, since the first expansion body 26 can be smoothly and compactly housed in the outer cylinder 22, the diameter of the outer cylinder 22 can be reduced as compared with a configuration where the first expansion body 26 is not curved and deformed into a heart shape.

Since the transfer instrument 11 includes the second carrier member 20 movable in the axial direction relative to the first carrier member 18, it is possible to transfer the medical sheet 300 from the first expansion body 26 to the recipient site 402 of the living body using the second carrier member 20 without using another device (forceps or the like). It is therefore possible to efficiently transfer the medical sheet 300 to the recipient site 402.

As illustrated in FIG. 19, in a state where the first expansion body 26 is housed in the outer cylinder 22, the distal portion of the second shaft 48 and the first expansion body 26 are brought into contact with each other by the movement restriction portion 23, and the relative movement of the first shaft 24 and the second shaft 48 in the axial direction is prevented. As a result, even if the user erroneously attempts to move the second carrier member 20 in the distal direction (the direction of the arrow X1) before the first expansion body 26 reaches the second position where the first expansion body 26 protrudes from the distal portion of the outer cylinder 22, the second carrier member 20 does not move relative to the first carrier member 18 in the axial direction. Specifically, even if the user erroneously grips and pushes the second carrier member 20 in the distal direction, both the first carrier member 18 and the second carrier member 20 do not move in the distal direction with respect to the outer cylinder 22. When the user erroneously grips and pushes the second carrier member 20 in the distal direction, however, the first carrier member 18 may also move in the distal direction with respect to the outer cylinder 22 together with the second carrier member 20. Also in this case, the second carrier member 20 does not move relative to the first carrier member 18 in the axial direction. As a result, the second expansion body 50 and the medical sheet 300 are prevented from protruding from the distal-end opening 80 of the outer cylinder 22. Consequently, the medical sheet 300 housed in the outer cylinder 22 is prevented from being pushed and damaged by the distal portion of the second shaft 48. Therefore, the medical sheet 300 can be efficiently transferred to the recipient site 402 without being damaged.

The movement restriction portion 23 is the pressure-application portion 56 constituting a distal portion of the second shaft 48. In a state where the first expansion body 26 is housed in the outer cylinder 22, the pressure-application portion 56 presses the first expansion body 26 against the lumen 78 (inner surface) of the outer cylinder 22. As a result, since the pressure-application portion 56 constituting the movement restriction portion 23 and the first expansion body 26 are brought into close contact with each other in a radial direction, the relative displacement between the first expansion body 26 and the second shaft 48 is restricted. The second shaft 48 can be moved in the axial direction with respect to the first shaft 24 in a state where the first expansion body 26 protrudes from the distal portion of the outer cylinder 22.

Since the pressure-application portion 56 is formed of an elastic body, the pressure-application portion 56 can be effectively brought into close contact with the first expansion body 26. Even if the second shaft 48 is pushed in the distal direction (the direction of the arrow X1) when the pressure-application portion 56 is housed in the outer cylinder 22, the second shaft 48 is bent and the pressing force is less likely to be transmitted to the pressure-application portion 56, and therefore the user easily notices the erroneous operation.

In the cross section orthogonal to the axis of the second shaft 48, the pressure-application portion 56 has the pair of linear portions 621 and 622 and the pair of convex portions 641 and 642 provided on both sides of the pair of linear portions 621 and 622. The first length D1 along the extending direction of the linear portions 621 and 622 is larger than the second length D2 (see FIG. 6). As a result, when the first expansion body 26 is housed in the outer cylinder 22, the first expansion body 26 is pushed toward the outer cylinder 22 by the pair of convex portions 641 and 642 provided on both sides of the linear portions 621 and 622, and the first expansion body 26 and the carrier holding portion 54 can be brought into closer contact with each other.

As illustrated in FIG. 2, the second expansion body 50 having a sheet shape and having the second support surface 74 capable of holding (configured to hold) the medical sheet 300 is provided at the distal portion of the second shaft 48. The second expansion body 50 is movable relative to the first expansion body 26 between the retracted position where the second expansion body 50 overlaps the first support surface 261 and the advanced position located further in the distal direction (the direction of the arrow X1) than the first support surface 261. With this configuration, the medical sheet 300 can be moved along the first support surface 261 of the first expansion body 26 by the second expansion body 50 including the second support surface 74.

As illustrated in FIG. 18, at the housed position (first position), the second expansion body 50 is housed in the outer cylinder 22 together with the first expansion body 26 with the second expansion body 50 deformed into a curved shape. With this configuration, even the second expansion body 50 having a width dimension can suitably house the second expansion body 50 together with the first expansion body 26 inside the outer cylinder 22 in the housing step.

The carrier holding portion 54 that holds the proximal portion of the second expansion body 50 is provided at the distal portion of the second shaft 48. Unlike the present embodiment, when the carrier holding portion 54 does not have a vertically symmetrical shape and the second expansion body 50 is held at the lower end of the carrier holding portion 54, the user rotates the second shaft 48 to reverse a vertical direction of the second expansion body 50, and the second expansion body 50 is disposed on the carrier holding portion 54. In this case, the second expansion body 50 rises with respect to the first support surface 261 of the first expansion body 26, and the first expansion body 26 and the second expansion body 50 are separated from each other in the vertical direction.

In the present embodiment, on the other hand, the carrier holding portion 54 is formed in line symmetry (vertical symmetry) with respect to the center line C of the carrier holding portion 54 as illustrated in FIG. 6 in the cross section orthogonal to the axis of the second shaft 48. That is, the cross-section of the carrier holding portion 54 is symmetrical about a horizontal line passing through the center line C so that portions of the cross-section vertically above and below such horizontal line are symmetrical. In addition, the carrier holding portion 54 holds the second expansion body 50 on center line C. Therefore, even when the user rotates the second shaft 48 to reverse the vertical direction of the second expansion body 50 (so that the surface of the second expansion body 50 facing the first support surface 261 is rotated 180° to face away from the first support surface 261), it is possible to suppress rising of the second expansion body 50 from the first support surface 261 of the first expansion body 26 as compared with a configuration in which the second expansion body 50 is held at (connected to) the lower end of the carrier holding portion 54. That is, if the user rotates the second shaft 48 so that the surface of the second expansion body 50 facing the first support surface 261 is rotated 180° to face away from the first support surface 261, the second expansion body 50 does not rise or move away from the first support surface 261 of the first expansion body 26. As a result, the medical sheet 300 can be stably transplanted by the second expansion body 50.

In the cross section orthogonal to the protruding direction of the first protrusion 38 and orthogonal to the first support surface 261, the cross section of each of the first protrusions 38 has a shape in which the intermediate portion 443 between the free end 442 and the fixed end 441 of the first protrusion 38 protrudes in a convex shape in the direction away from the first support surface 261. With this configuration, when the first expansion body 26 is housed in the outer cylinder 22, the first expansion body 26 can be reliably curved and deformed into a heart shape. As a result, it is possible to prevent the medical sheet 300 from being damaged due to the deformation of the first expansion body 26 into a shape other than the heart shape.

Unlike the present embodiment, when the intermediate portion 443 has a curved shape in such a way as to be recessed toward the first support surface 261 on the back surface 462 of the first expansion body 26, the first expansion body 26 is on rare occasions not curved and deformed into an appropriate heart shape in the outer cylinder 22. For example, since the free end 442 (front surface 461) of one first protrusion 38 rides on the fixed end 441 (back surface 462) of the other first protrusion 38 that is curved and deformed, the free end 442 is on rare occasions bent in a V shape toward the inner surface of the outer cylinder 22. In the present embodiment, since the intermediate portion 443 has a shape bulging in a convex shape in the direction away from the first support surface 261, it is possible to prevent V-shaped bending of the first expansion body 26.

By forming the intermediate portions 443 of the pair of first protrusions 38 in arc shapes, the first protrusions 38 having shape bulging outward can be easily formed.

Since a curvature R of the intermediate portion 443 of each first protrusion 38 increases in the proximal direction (the direction of the arrow X2) of the first expansion body 26, the interval W in the width direction between the first protrusions 38 on the distal side of the first protrusion 38 can be increased. As a result, the medical sheet 300 can be easily placed on the first support surface 261 of the first expansion body 26 through the distal-end support portion 42.

As illustrated in FIG. 4, since the interval W between the pair of bent portions 444 in the width direction of the first expansion body 26 decreases in the proximal direction, when the first expansion body 26 is housed in the outer cylinder 22 from the proximal side, the pair of first protrusions 38 can be suitably curved and deformed in a conical shape by contact with the outer cylinder 22, and accordingly, interference between the proximal ends of the pair of bent portions 444 is suppressed, so that the first expansion body 26 can be smoothly housed in the outer cylinder 22 from the proximal side.

Since the distal portion of the first expansion body 26 has an arcuate shape connecting the pair of second protrusions 40 when viewed from a direction orthogonal to the first support surface 261, sliding resistance between the first expansion body 26 and the outer cylinder 22 can be reduced when the first expansion body 26 is housed in the outer cylinder 22 as compared with a configuration in which the distal portion of the first expansion body 26 has a shape having corners. When the medical sheet 300 is placed on the first expansion body 26 by moving the first expansion body 26 in the distal direction (the direction of the arrow X1), the operation can be smoothly performed by gradually inserting the first expansion body 26 into an interface between the medical sheet 300 and the Petri dish 401 (container).

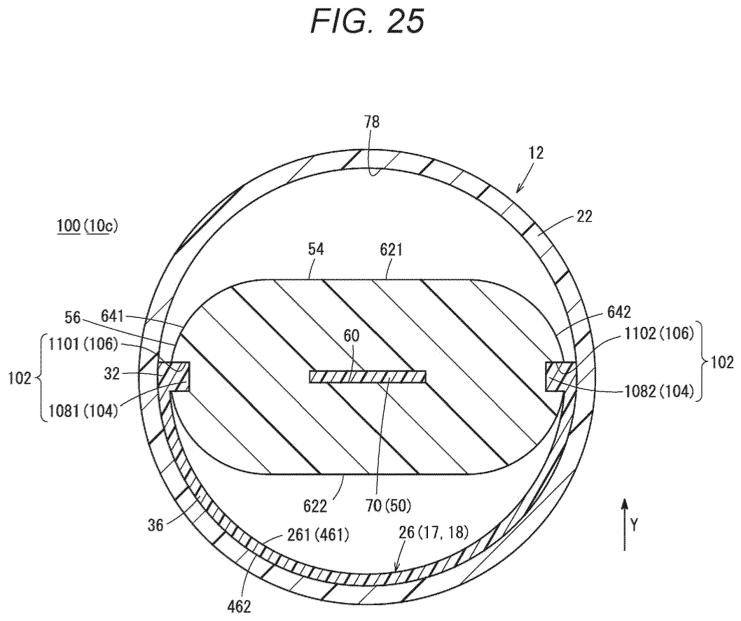

As illustrated in FIG. 25, a transfer instrument 100 according to a first modification includes movement restriction portions 102. In the present modification, the same components as those of the above-described transfer instrument 11 are identified by the same reference numerals, and a detailed description of such components is not repeated. The same applies to transfer instruments 120, 130, 150 and 160 according to second to fifth modifications described later.

The movement restriction portions 102 each include a first fitting portion 104 provided on the first expansion body 26 and a second fitting portion 106 provided on the carrier holding portion 54 at the distal portion of the second shaft 48.

The first fitting portions 104 have a pair of first convex portions 1081 and 1082 protruding upward from the first support surface 261 of the first expansion body 26. The first convex portions 1081 and 1082 are disposed at an equal distance from the center of the first expansion body 26 in the width direction. When viewed from a direction orthogonal to the width direction of the first expansion body 26 (such as shown in FIG. 25), cross sections of the first convex portions 1081 and 1082 are, for example, rectangular. The cross sections of the first convex portions 1081 and 1082 may have another shape (a semicircular shape or the like), instead.

The second fitting portions 106 are disposed at both ends of the carrier holding portion 54 (pressure-application portion 56) in the width direction (convex portions 641 and 642). The second fitting portions 106 have concave portions 1101 and 1102 recessed from outer surfaces of the convex portions 641 and 642. The concave portions 1101 and 1102 are recessed in the width direction from the outer surfaces of the convex portions 641 and 642. When viewed from the axial direction of the carrier holding portion 54, cross sections of the concave portions 1101 and 1102 have rectangular shapes corresponding to the first convex portions 1081 and 1082. The cross sections of the concave portions 1101 and 1102 may have another shape (a semicircular shape or the like), instead.

In a state where the first expansion body 26 is housed in the outer cylinder 22 and the first expansion body 26 is curved and deformed, the first convex portions 1081 and 1082 of the first fitting portions 104 constituting the movement restriction portions 102 are fitted into the concave portions 1101 and 1102 of the second fitting portions 106, respectively. As a result, the relative displacement between the first expansion body 26 and the carrier holding portion 54 is restricted, and accordingly the relative movement of the second shaft 48 in the axial direction with respect to the first shaft 24 is restricted.

As described above, in the transfer instrument 100 according to the first modification, in the state where the first expansion body 26 is housed in the outer cylinder 22 (first position), the first fitting portions 104 and the second fitting portions 106 in the movement restriction portions 102 are fitted to each other, so that the relative movement of the first shaft 24 including the first expansion body 26 and the second shaft 48 in the axial direction is prevented. When the fitting between the first fitting portions 104 and the second fitting portions 106 of the movement restriction portions 102 is released in the state where the first expansion body 26 protrudes from the distal portion of the outer cylinder 22 (second position), the second shaft 48 becomes movable in the distal direction (the direction of the arrow X1) with respect to the first expansion body 26.

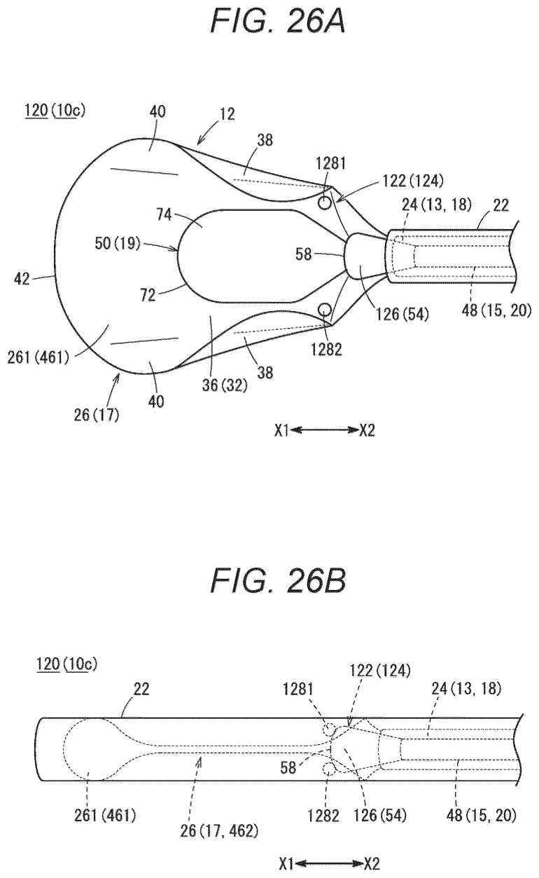

As illustrated in FIG. 26A, the transfer instrument 120 according to the second modification includes a movement restriction portion 122. The movement restriction portion 122 includes a first engagement portion 124 and second engagement portions 126. The first engagement portion 124 is provided on the surface 461 of the first expansion body 26 including the first support surface 261. The second engagement portions 126 are configured by the carrier holding portion 54 at the distal portion of the second shaft 48.

The first engagement portion 124 has a pair of second convex portions 1281 and 1282 protruding upward from the first support surface 261. The second convex portions 1281 and 1282 are disposed at an equal distance from the center of the first expansion body 26 in the width direction. The second engagement portions 126 are disposed on both sides of the distal end of the carrier holding portion 54.

As illustrated in FIG. 26B, in a state where the first expansion body 26 is housed in the outer cylinder 22 and the first expansion body 26 is curved and deformed, the second convex portions 1281 and 1282 of the first engagement portion 124 and the second engagement portions 126 face each other in the axial direction of the outer cylinder 22, and the first engagement portion 124 and the second engagement portions 126 are engaged with each other in the axial direction.

As described above, in the transfer instrument 120 according to the second modification, in the state (first position) where the first expansion body 26 is housed in the outer cylinder 22 illustrated in FIG. 26B, the first engagement portion 124 and the second engagement portions 126 constituting the movement restriction portion 122 are engaged in the axial direction, so that the relative movement of the first shaft 24 including the first expansion body 26 and the second shaft 48 in the axial direction is prevented. When the first expansion body 26 is unfolded in the state (second position) in which the first expansion body 26 illustrated in FIG. 26A protrudes from the distal-end opening 80 of the outer cylinder 22, the first engagement portion 124 and the second engagement portions 126 do not face each other in the axial direction, and the engagement between the first engagement portion 124 and the second engagement portions 126 is released. As a result, the second shaft 48 can move in the distal direction (the direction of the arrow X1) with respect to the first expansion body 26.

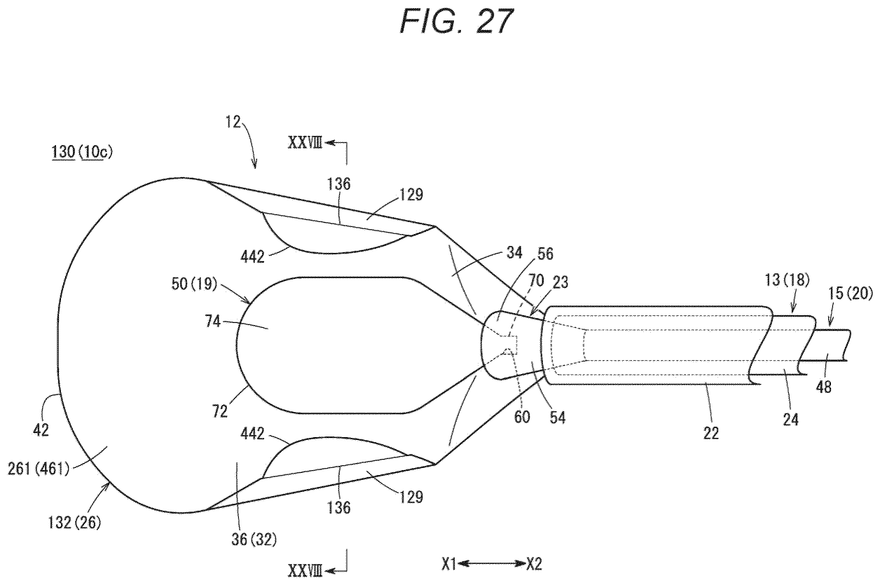

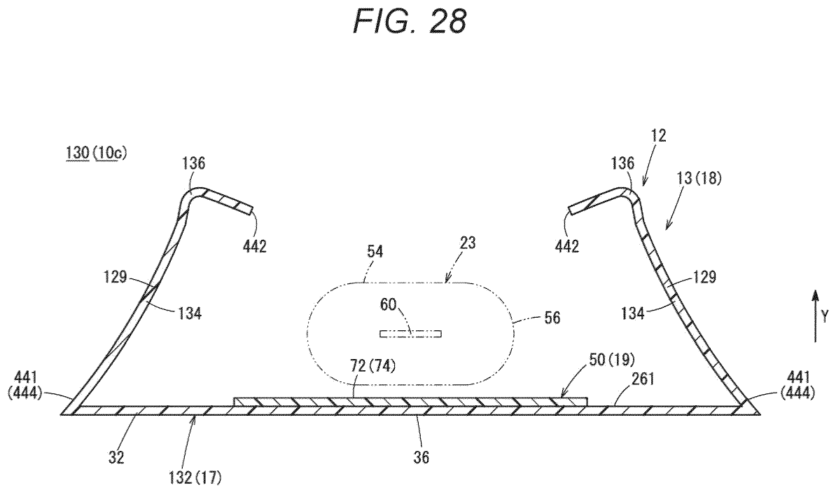

As illustrated in FIGS. 27 and 28, the transfer instrument 130 according to the third modification includes a first support portion 132. Protrusions 129 provided on both sides of the first support portion 132 in the width direction each have an intermediate portion 134 between a free end 442 and a fixed end 441. The intermediate portion 134 has a shape bulging in a convex shape in a direction away from the first support surface 261. The intermediate portion 134 has an intermediate bent portion 136 bent in such a way as to form a mountain fold on the back surface 462. The intermediate bent portion 136 is disposed closer to the free end 442 in the intermediate portion 134.

As described above, in the transfer instrument 130 according to the third modification, by providing the intermediate bent portion 136 in each protrusion 129 of the first support portion 132, it is possible to easily form the protrusion 129 having a shape bulging in a convex shape in the direction away from the first support surface 261.

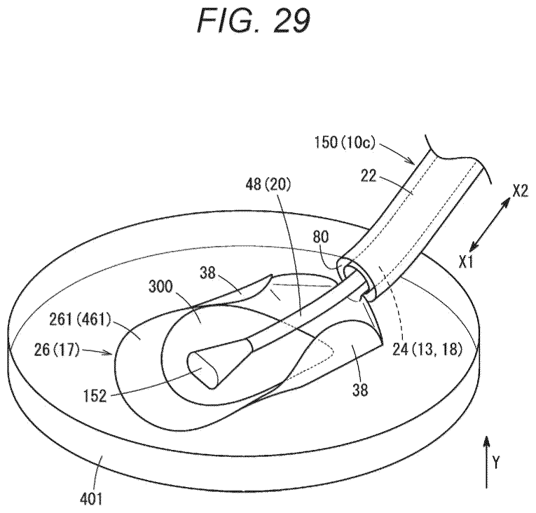

Although the case where the second expansion body 50 is attached to the distal portion of the second shaft 48 has been described, the medical device is not limited thereto. For example, as in the transfer instrument 150 according to the fourth modification illustrated in FIG. 29, a pressing body 152 may be provided at the distal portion of a second shaft 48. The pressing body 152 has, for example, a flat cross-sectional shape having a flat bottom surface. The medical sheet 300 placed on the first support surface 261 of the first expansion body 26 can be pressed downward by the bottom surface of the pressing body 152 to push out the medical sheet 300 in the distal direction (the direction of the arrow X1) along the first support surface 261 of the first expansion body 26.

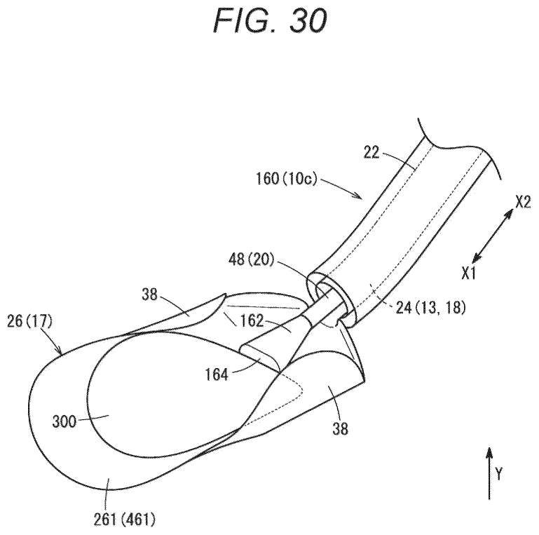

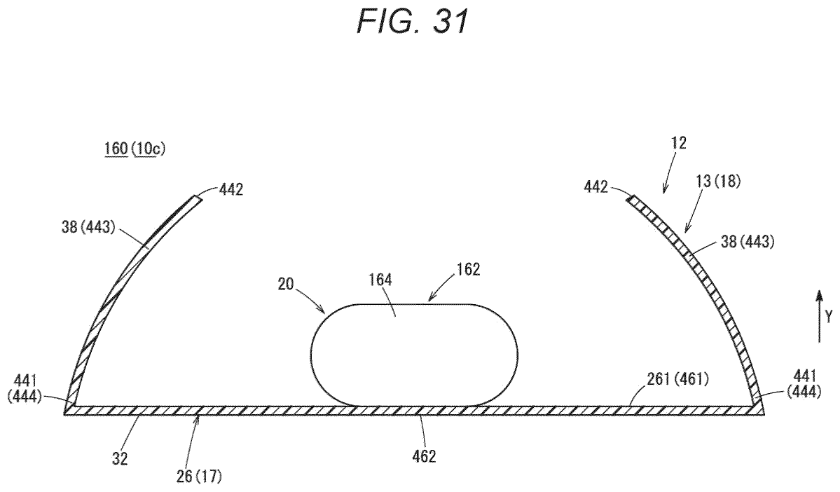

As in the transfer instrument 160 according to the fifth modification illustrated in FIGS. 30 and 31, a pressing body 162 may be provided at the distal portion of the second shaft 48. A pressing surface 164 is provided at a distal end of the pressing body 162. The pressing surface 164 is a flat surface orthogonal to an axis of the pressing body 162. A proximal end of the pressing body 162 is connected to the distal portion of the second shaft 48. The medical sheet 300 placed on the first support surface 261 of the first expansion body 26 can be pressed by the pressing surface 164 of the pressing body 162 to push out the medical sheet 300 in the distal direction (the direction of the arrow X1) along the first support surface 261 of the first expansion body 26.

The detailed description above describes embodiments of a medical device holding structure representing examples of the new medical device holding structure disclosed here. The invention is not limited, however, to the precise embodiments and variations described. Various changes, modifications and equivalents can be effected by one skilled in the art without departing from the spirit and scope of the invention as defined in the accompanying claims. It is expressly intended that all such changes, modifications and equivalents that fall within the scope of the claims are embraced by the claims.

What is claimed is:

1. A medical device holding structure comprising:
a medical device and a mount;
the medical device including an elongated shaft having a distal portion, the medical device also including an expandable and contractible expansion body positioned at the distal portion of the shaft and extending away from the distal portion of the elongated shaft;
the mount having oppositely disposed first and second surfaces, one end portion of the mount being folded back upon itself so that the first surface of the mount faces a housing portion, the one end portion of the mount that is folded back upon itself defining a cover portion of the mount, the mount including an elongated mount body that extends from the cover portion to an opposite end of the mount that is opposite the one end portion;
the elongated shaft of the medical device overlying the first surface of the mount so that the first surface of the mount in the elongated mount body faces the elongated shaft while the second surface of the mount in the elongated mount body faces away from the elongated shaft;
the expandable and contractible expansion body being positioned in the housing portion so that the expandable and contractible expansion body is covered by the cover portion.

2. The medical device holding structure according to claim 1, further comprising an upstanding first support piece having a fixed end that is fixed to the elongated mount body, the upstanding first support piece extending away from the first surface of the mount, the upstanding first support piece including a support hole that passes through the upstanding first support piece in a thickness direction of the upstanding first support piece, a portion of the elongated shaft of the medical device being positioned in the support hole.

3. The medical device holding structure according to claim 2, wherein the upstanding first support piece is positioned, relative to a longitudinal extent of the mount, between the cover portion and the opposite end of the mount.

4. The medical device holding structure according to claim 1, further comprising an upstanding first support piece and an upstanding second support piece that each have a fixed end that is fixed to the elongated mount body, the upstanding first support piece and the upstanding second support piece extending away from the first surface of the mount, the upstanding first support piece including a support hole that passes through the upstanding first support piece in a thickness direction of the upstanding first support piece, the upstanding second support piece including a support hole that passes through the upstanding second support piece in a thickness direction of the upstanding second support piece, a portion of the elongated shaft of the medical device being positioned in the support hole in the upstanding first support piece and in the support hole in the upstanding second support piece.

5. The medical device holding structure according to claim 4, wherein the upstanding first support piece and the upstanding second support piece are spaced apart rom one another along the elongated mount body and are positioned between cover portion and the opposite end of the mount.

6. The medical device holding structure according to claim 3, further comprising a plurality of hooks each having a fixed end that is fixed to the elongated mount body, each of the hooks overlying a part of the elongated shaft of the medical device, the hooks being spaced apart from one another along the elongated mount body, the hooks being arranged on opposite sides of the elongated shaft in a width direction of the elongated mount body.

7. The medical device holding structure according to claim 1, wherein the mount includes a perforation that extends in a width direction of the elongated mount body, the perforation being located to separate the cover portion from the elongated mount body.

8. The medical device holding structure according to claim 1, wherein the cover portion includes an opening that passes through the cover portion to permit visual recognition of the expansion body from outside the cover portion.

9. A medical device holding structure comprising:
a medical device; and
a mount that holds the medical device;
the medical device including a shaft and an expandable and contractible expansion body disposed at a distal portion of the shaft;
the mount including a mount body and a cover portion,
the mount having a holding surface at which is held the medical device;
the cover portion being continuous with the mount body and covering the expansion body of the medical device;
the cover portion having a bent shape folded back in such a way as to cover the expansion body; and
an inside of the cover portion forming a housing portion in which is housed the expansion body.

10. The medical device holding structure according to claim 9, wherein
the mount includes an upstanding support piece projecting away from the holding surface of the mount body and having a support hole in which is positioned a portion of the medical device so that the medical device is held on the mount, and
the cover portion is configured to be unfolded in a direction away from the expansion body so that the support piece tilts toward the holding surface.

11. The medical device holding structure according to claim 9, wherein the medical device is a first medical device, the shaft is a first shaft and the expandable and contractible expansion body is a first expandable and contractible expansion body, and further comprising a second medical device that includes a second shaft and a second expandable and contractible expansion body disposed at a distal portion of the second shaft, the mount including a plurality of device holding portions each configured to hold a respective one of the first and second medical devices, each of the plurality of device holding portions including at least one upstanding support piece projecting away from the holding surface of the mount body and having a support hole in which is positioned a portion of a respective one of the respective medical device so that the respective medical devices are held on the mount, the cover portion being configured to be unfolded in a direction away from the expansion body so that the support pieces tilt toward the holding surface.

12. The medical device holding structure according to claim 11, further comprising:
a separation portion configured to separate one device holding portion and another device holding portion between two adjacent device holding portions among the plurality of device holding portions.

13. The medical device holding structure according to claim 9, wherein
the mount includes a cover separation portion configured to separate the cover portion from the mount body, and
the cover separation portion has a break line for breaking the cover portion from the mount body.

14. The medical device holding structure according to claim 10, wherein
the cover portion has an opening penetrating the cover portion, and the expansion body is visually recognizable from an outside of the cover portion through the opening.

15. The medical device holding structure according to claim 9, wherein
the medical device includes an outer cylinder, and an inner member disposed inside the outer cylinder in such a way as to be movable along an axial direction of the outer cylinder,
the inner member includes the shaft positioned in the outer cylinder,
the expansion body is housed in the outer cylinder in a contracted state at a first position of the expansion body,
the expansion body is unfolded from the contracted state at the first position to an expanded state at a second position by moving the inner member in a distal direction relative to the outer cylinder so that the expansion body is exposed from the outer cylinder and protrudes from a distal-end opening of the outer cylinder, and
the expansion body of the medical device is covered with the cover portion in a state where the inner member is located at the second position with respect to the outer cylinder and the expansion body is unfolded.

16. The medical device holding structure according to claim 15, wherein
the expansion body is a flexible sheet having a front support surface configured to hold a medical sheet and a back surface opposite to the front support surface,
the expansion body includes a pair of protrusions protruding upward from both sides, in a width direction, of the support surface orthogonal to a moving direction of the shaft, and
at the first position, the expansion body is housed in the outer cylinder in a state of being curved and is deformed such that both sides, in the width direction, of the expansion body on the back surface come into contact with each other.

17. A mount for holding a medical device that includes a shaft and an expansion body disposed at a distal portion of the shaft, the mount comprising:
a mount body having a holding surface for holding the medical device;
a cover portion that is continuous with the mount body and that is configured to cover the expansion body of the medical device;
the cover portion being configured to be folded back on itself in a state where the medical device is held by the mount body, so that the cover portion covers the expansion body of the medical device.

18. The mount according to claim 17, further comprising:
a plurality of device holding portions configured to hold a plurality of the medical devices, wherein the plurality of device holding portions includes a plurality of support pieces.

19. The mount according to claim 18, further comprising:
a separation portion that can be used to separate one device holding portion and another device holding portion from each other between two adjacent device holding portions among the plurality of device holding portions.

20. The mount according to claim 17, further comprising:
a cover separation portion configured to separate the cover portion from the mount body, wherein
the cover separation portion has a break line for breaking the cover portion from the mount body.
